(19) United States
(12) Patent Application Publication
(10) Pub. No.: US 2020/0012959 A1
(43) Pub. Date: Jan. 9, 2020

(54) SYSTEMS AND TECHNIQUES FOR IDENTIFYING AND EXPLOITING RELATIONSHIPS BETWEEN MEDIA CONSUMPTION AND HEALTH

(71) Applicant: Bose Corporation, Framingham, MA (US)

(72) Inventors: Ketki Karanam, Newton, MA (US); Alexis Kopikis, Needham, MA (US); Vikram Krishnan, Boston, MA (US); Nilesh N. Kuchekar, Somerville, MA (US); Abby B. Marsh, Concord, MA (US); W. Edward Martucci, Westwood, MA (US); Steven A. Sian, Auburndale, MA (US)

(73) Assignee: Bose Corporation, Framingham, MA (US)

(21) Appl. No.: 16/573,597

(22) Filed: Sep. 17, 2019

Related U.S. Application Data
(62) Division of application No. 15/721,763, filed on Sep. 30, 2017.
Application No. 15/721,763 is a continuation of application No. 14/831,540, filed on Aug. 20, 2015, now abandoned.
(60) Provisional application No. 62/130,964, filed on Mar. 10, 2015.
(60) Provisional application No. 62/039,745, filed on Aug. 20, 2014.

(51) Int. Cl. G06N 5/04; G16H 50/20; G16H 50/30; A61B 5/0205; A61B 5/00; A61B 5/16; A61M 21/00

(52) U.S. Cl. CPC G06N 5/048; G16H 50/20; G16H 50/30; A61B 5/02055; A61B 5/7246; A61B 5/1118; A61M 21/00; A61B 2560/0242; A61B 5/681; A61B 5/6898; A61B 5/4815; A61B 5/165

(57) ABSTRACT

A system for inducing a physical state in a user. The system includes a sensor associated with the user, an audio output device, and a computer. The computer obtains, from a database, an identification of a type of music associated with a first phase of sleep, and determines, based on data from the sensor, that a user is in a second phase of sleep. Music of the type associated with the first phase of sleep is selected, and the selected music is output to the user through the audio output device.

### CROSS-REFERENCE TO RELATED APPLICATIONS

This application is a divisional of co-pending U.S. patent application Ser. No. 15/721,763, filed on Sep. 30, 2017, which is a continuation of U.S. patent application Ser. No. 14/831,540, filed on Aug. 20, 2015, which claims priority and benefit under 35 U.S.C. § 119(e) of U.S. Provisional Patent Application No. 62/039,745, filed on Aug. 20, 2014, and U.S. Provisional Patent Application No. 62/130,964, filed on Mar. 10, 2015. The foregoing applications are hereby incorporated by reference to the maximum extent permitted by applicable by law.

### FIELD OF INVENTION

The present disclosure relates generally to systems and techniques for identifying and exploiting relationships between media consumption and health.

### BACKGROUND

Media consumption plays a major role in the everyday lives of many people, with some people spending on average approximately 64 hours/week attending to various audio and visual media (Rentfrow P. J. et al, 2011, J. Pers. 79(2): 223-258). People consume media as a primary activity or as a secondary activity associated with other activities including, for example, exercise, driving or building concentration on a task.

Medical diagnostic processes are used to determine whether a patient has a health condition (e.g., a disease, disorder, illness, chronic condition, etc.). In general, medical diagnostic processes involves determining the patient's health state (e.g., physiological state, psychological state, etc.), identifying symptoms or signs of health conditions, and determining which health condition(s) explain the patient's health state and symptoms/signs.

Biomarkers can be used to determine the health state of a patient. The presence of certain “diagnostic biomarkers” in a patient can be a sign of health condition in a patient. For example, the presence of certain autoantibodies in a patient's blood is a biomarker for some autoimmune diseases. The presence of certain “predictive biomarkers” in a patient can indicate how the patient is likely to respond to a particular treatment for a health condition. For example, the presence of the KRAS mutations in a patient's cancer cells is predictive of the patient's resistance to certain cancer therapies.

A Summary of the subject matter of the present disclosure is provided below, followed by a Detailed Description of some embodiments. The Detailed Description also describes the motivations underlying some embodiments.

### SUMMARY

According to an aspect of the present disclosure, a method is provided, including obtaining media consumption data regarding media content consumed by a user during one or more time periods, wherein the media content includes a plurality of media content items having one or more same features, obtaining health data, wherein at least a portion of the health data relates to health states of the user during the one or more time periods, synchronizing the media consumption data and the health data, determining a strength of a relationship between a health state of the user and consumption by the user of media content having the one or more features, based at least in part on portions of the media consumption data corresponding to user consumption of the plurality of media content items having the one or more features and on the synchronized health data, and predicting an effect of consuming a media content item on the user's health, wherein the prediction is based at least in part on a determination that the strength of the relationship between the health state of the user and the consumption by the user of media content having the one or more features exceeds a threshold strength. The method may be performed by one or more computers. Other embodiments of this aspect include systems, apparatus, computer programs, and computer-readable media.

These and other aspects can optionally include one or more of the following characteristics. In some embodiments, obtaining the media consumption data includes receiving the media consumption data from one or more sensors configured to detect media content and/or from one or more devices configured to present media content. In some embodiments, the media consumption data includes preference data indicating one or more preferences of the user regarding the media content consumed by the user, and the determination that the strength of the relationship between the health state of the user and the consumption by the user of the media content having the one or more features exceeds the threshold strength is based, at least in part, on the one or more preferences of the user.

In some embodiments, the one or more features relate to sound quality of an audio portion of the media content. In some embodiments, the one or more features include timbre, pitch, key, and/or mode. In some embodiments, the one or more features relate to harmonic complexity of an audio portion of the media content. In some embodiments, the one or more features include pitch, key, and/or mode. In some embodiments, the one or more features include one or more low-level audio features of an audio portion of the media content. In some embodiments, the one or more low-level audio features include Mel-Frequency Cepstral Coefficients (MFCC), Audio Spectrum Envelope (ASE), Audio Spectrum Flatness (ASF), Linear Predictive Coding Coefficients, Zero Crossing Rate (ZCR), Audio Spectrum Centroid (ASC), Audio Spectrum Spread (ASS), spectral centroid, spectral rolloff, and/or spectral flux. In some embodiments, the one or more features include a compound feature, and the compound feature includes a combination of one or more low-level audio features of an audio portion of the media content, one or more features relating to sound quality of an audio portion of the media content, and/or one or more features relating to harmonic complexity of an audio portion of the media content.

In some embodiments, obtaining the health data includes receiving the health data from one or more sensors configured to sense health parameters of the user. In some embodiments, the health data includes values of one or more health parameters of the user, and the one or more health parameters relate to the user's physiology, psychology, mood, activity, well-being, and/or behavior.

In some embodiments, the method further includes obtaining context data, wherein at least a portion of the context data relates to contexts of the user during the one or more time periods, and synchronizing the context data with the media consumption data and the health data, wherein the determination that the strength of the relationship between the health state of the user and the consumption by the user of the media content having the one or more features exceeds the threshold strength is based, at least in part, on the context data.

In some embodiments, the media content consumed by the user during the one or more time periods does not include the media content item for which the prediction is made. In some embodiments, the media content item has the one or more features. In some embodiments, the predicted effect of consuming the media content item includes a predicted long-term effect of consuming the media content item.

In some embodiments, the prediction is further based at least in part on a determination that a strength of a relationship between the health state in a population and consumption of media content having the one or more features exceeds a threshold strength, and the population includes other users. In some embodiments, the prediction is further based at least in part on one or more user preferences relating to the media content and/or to the one or more features of the media content. In some embodiments, the predicted effect on the user's health includes a predicted change in the user's purchasing intent.

In some embodiments, the method further includes attaching a health tag to the media content item as metadata of the media content item, wherein the health tag indicates the predicted effect of consuming the media content item on the user's health. In some embodiments, the method further includes predicting an effect of consuming the media content item on a population's health, wherein the prediction of the effect on the population's health is based at least in part on a determination that a strength of a relationship between the health state in the population and consumption of media content having the one or more features exceeds a threshold strength, and wherein the population includes other users, and attaching a health tag to the media content item as metadata of the media content item, wherein the health tag indicates the predicted effect of consuming the media content item on the population's health. In some embodiments, the predicted effect on the population's health includes a predicted change in the population's purchasing intent.

In some embodiments, the method further includes receiving data identifying a target health state of the user, selecting one or more media content items, wherein a predicted effect of the user consuming the one or more media content items includes the user's health attaining the target health state, and recommending the one or more media content items for consumption by the user. In some embodiments, the selection of the one or more media content items is based, at least in part, on health tags associated with the one or more media content items. In some embodiments, the selection of the one or more media content items is based, at least in part, on a strength of a relationship between features of the one or more media content items and the health state. In some embodiments, the media consumption data includes pattern data regarding one or more patterns of media consumption by the user, and the selection of the one or more media content items is based, at least in part, on a strength of a relationship between the health state and a pattern of media consumption by the user.

In some embodiments, the method further includes obtaining media biomarker data, wherein the media biomarker data includes a media consumption signature associated with a health condition, determining whether the media consumption signature associated with the health condition matches the media consumption data for the user, and diagnosing the user with the health condition based, at least in part, on a determination that the media consumption signature associated with the health condition matches the media consumption data for the user. In some embodiments, the prediction of the effect of consuming the media content item on the user's health is further based, at least in part, on the determination that the media consumption signature associated with the health condition matches the media consumption data for the user. In some embodiments, the method further includes prescribing, based at least in part on the determination that the media consumption signature associated with the health condition matches the media consumption data for the user, a therapy for the user. In some embodiments, the prescribed therapy includes consuming particular items of media content.

In some embodiments, the health data are first health data, the media consumption data are first media consumption data, and obtaining the media biomarker data includes obtaining second health data regarding health of a population, obtaining second media consumption data regarding media consumption of the population, wherein the second media consumption data include the media consumption signature, generating relationship data regarding a relationship between the media consumption signature and a portion of the second health data corresponding to the health condition, determining whether a strength of the relationship exceeds a threshold strength, and generating the media biomarker data based, at least in part, on a determination that the strength of the relationship between the media consumption signature and the portion of the second health data corresponding to the health condition exceeds the threshold strength. In some embodiments, determining whether the media consumption signature associated with the health condition matches the media consumption data for the user includes determining, based on the media consumption data, one or more media consumption signatures of the user, and comparing the media consumption signature associated with the health condition to the one or more media consumption signatures of the user.

In some embodiments, the method further includes mapping at least a portion of the media consumption data and at least a portion of the health data to values of one or more sensory parameters, wherein the portion of the media consumption data and the portion of the health data correspond to a same time period, and presenting sensory information to the user, wherein the sensory information represents the values of the one or more sensory parameters. In some embodiments, the sensory information includes visual information, auditory information, tactile information, olfactory information, and/or taste information. In some embodiments, mapping at least the portion of the media consumption data and at least the portion of the health data to the values of one or more sensory parameters includes mapping at least the portion of the media consumption data to a first visualization parameter and mapping at least the portion of the health data to a second visualization parameter. In some embodiments, the first visualization parameter includes a color, transparency, shape, rotation, and/or pixilation of a graphic, the second visualization parameter includes a color, transparency, shape, rotation, and/or pixilation of a graphic, and the first visualization parameter differs from the second visualization parameter. In some embodiments, the health data includes values of a first health parameter for the user, the one or more sensory parameters include a first sensory parameter, and mapping at least the portion of the media consumption data and at least the portion of the health data to the values of the one or more sensory parameters includes generating correlation data regarding a correlation between the portion of the media consumption data and the values of the first health parameter, and mapping the correlation data to the first sensory parameter.

According to another aspect of the present disclosure, a method is provided, including obtaining media biomarker data, wherein the media biomarker data includes a media consumption signature associated with a health condition, obtaining media consumption data regarding media consumption of a population, determining whether the media consumption signature associated with the health condition matches the media consumption data for the population, diagnosing the population with the health condition based, at least in part, on a determination that the media consumption signature associated with the health condition matches the media consumption data for the population, and communicating information associated with diagnosis of the health condition to a user. The method may be performed by one or more computers. Other embodiments of this aspect include systems, apparatus, computer programs, and computer-readable media.

These and other aspects can optionally include one or more of the following features. In some embodiments, In some embodiments, the population consists of an individual person. In some embodiments, the population includes a plurality of people. In some embodiments, the people have one or more characteristics in common.

In some embodiments, the population is a first population, the media consumption data are first media consumption data, and obtaining the media biomarker data includes obtaining health data regarding health of a second population, obtaining second media consumption data regarding media consumption of the second population, wherein the second media consumption data include the media consumption signature, generating relationship data regarding a relationship between the media consumption signature and a portion of the health data corresponding to the health condition, determining whether a strength of the relationship exceeds a threshold strength, and generating the media biomarker data based, at least in part, on a determination that the strength of the relationship between the media consumption signature and the portion of the health data corresponding to the health condition exceeds the threshold strength. In some embodiments, the media biomarker data is generated by a research tool. In some embodiments, the relationship between the media consumption signature and the portion of the health data corresponding to the health condition includes a correlation. In some embodiments, the first population and the second population are the same.

In some embodiments, the media consumption signature associated with the health condition includes an amount, rate, pattern, range of amounts, range of rates, or plurality of patterns of consumption of media content. In some embodiments, the media consumption signature associated with the health condition includes an amount, rate, range of amounts, or range of rates of media content within a media content category. In some embodiments, the media consumption signature associated with the health condition includes an amount, rate, pattern, range of amounts, range of rates, or plurality of patterns of consumption of media content including a feature having a value within a particular range.

In some embodiments, determining whether the media consumption signature associated with the health condition matches the media consumption data for the population includes determining, based on the media consumption data, one or more media consumption signatures of the population, and comparing the media consumption signature associated with the health condition to the one or more media consumption signatures of the population. In some embodiments, the diagnosis is further based, at least in part, on health data regarding health of the population. In some embodiments, communicating information associated with the diagnosis includes causing the information to be displayed, causing the information to be presented audibly, and/or transmitting the information.

In some embodiments, the method further includes predicting, based at least in part on the determination that the media consumption signature associated with the health condition matches the media consumption data for the population, an effect on a member of the population of consuming a particular item of media content. In some embodiments, the method further includes prescribing, based at least in part on the determination that the media consumption signature associated with the health condition matches the media consumption data for the population, a therapy for a member of the population. In some embodiments, the prescribed therapy includes consuming particular items of media content. In some embodiments, the prescribed therapy further includes administration of a drug or performance of a medical intervention in connection with the consumption of the particular items of media content.

In some embodiments, the method further includes attaching a health tag to a media content item as metadata of the media content item based, at least in part, on a determination that the media content item includes the media consumption signature associated with the health condition, wherein the health tag indicates that consumption of the media content item is associated with the health condition.

In some embodiments, the media consumption data corresponds to media consumption of the population during a first time period, the method further includes monitoring a status of the health condition in the population, and monitoring the status of the health condition includes obtaining second media consumption data regarding media consumption of the population during a second time period, and determining whether the media consumption signature associated with the health condition matches the second media consumption data for the population.

Details of one or more embodiments of the subject matter described in the present disclosure are set forth in the accompanying drawings and the description below. Other features, aspects, and advantages of the subject matter will become apparent from the description, the drawings, and the claims.

### BRIEF DESCRIPTION OF THE DRAWINGS

Certain advantages of some embodiments may be understood by referring to the following description taken in conjunction with the accompanying drawings. In the drawings, like reference characters generally refer to the same parts throughout the different views. Also, the drawings are not necessarily to scale, emphasis instead generally being placed upon illustrating principles of some embodiments.

FIG. 1 is a block diagram of a system for identifying and exploiting relationships between media consumption and health, according to some embodiments.

FIG. 2A shows a visualization of a user's media consumption data, health data, and context data, according to some embodiments.

FIG. 2B shows another visualization of a user's music consumption data, health data, and context data, in accordance with embodiments.

FIG. 3 is a block diagram of another system for identifying and exploiting relationships between media consumption and health, which includes a biofeedback module for providing biofeedback to the user to recommend and present suitable media content selected based on the user's health data, context data, and media consumption data, according to some embodiments.

FIGS. 4A, 4B, 4C, and 4D show visualizations of a user's media consumption data, health data, and context data, according to some embodiments.

FIGS. 5A and 5B show synchronized media consumption data and health data from an exemplary user of a system for providing personalized, therapeutic biofeedback to regulate cardiovascular parameters, according to some embodiments.

FIGS. 6, 7, 8, and 9 show exemplary visualizations of health data and/or media consumption data using two-dimensional geometry, in accordance with some embodiments.

FIGS. 10, 11, 12, and 13 show exemplary visualizations of health data and/or media consumption data using three-dimensional geometry, in accordance with some embodiments.

FIG. 14-15 show exemplary visualizations of health data and/or media consumption data using tunnels, in accordance with some embodiments.

FIGS. 16-17 show exemplary visualizations of health data and/or media consumption data using interaction between liquids, in accordance with some embodiments.

FIGS. 18-19 show exemplary visualizations of health data and/or media consumption data using musical paths, in accordance with some embodiments.

FIG. 20 is a flowchart of a method for identifying and exploiting relationships between media consumption and health, according to some embodiments.

FIG. 21 is a flowchart of another method for identifying and exploiting relationships between media consumption and health, according to some embodiments.

FIG. 22 is a block diagram of a computer for identifying and exploiting relationships between media consumption and health, according to some embodiments.

### DETAILED DESCRIPTION

Motivation for and Advantages of Some Embodiments

Besides its entertainment and educational value, media can have significant direct effects on human health (e.g., physiological health, emotional health, and/or behavioral health). As a specific example, music can affect the brain and body, including processes such as mood, memory processing, cardiovascular rhythms, stress and pain regulation and physical movement. The structure and composition of music and its acoustic properties can mediate distinct physiological and psychological effects. Listening to music can have therapeutic benefits for various health conditions. These therapeutic effects can potentially be enhanced by selecting music that is aligned with an individual's personal music associations and preferences. Therefore, presenting a listener music that is selected based on its acoustic patterns and the listener's personalized health and contextual parameters has the potential to effect specific mood and health benefits for the listener and improve his/her general well-being.

While subjective evaluations (e.g., user-provided lists of media content items that a user likes/dislikes in specific settings) can capture user mood and preference in a limited context, they are not always efficient at or appropriate for selecting media content items for eliciting a desired health effect. For example, music recommendation engines that generate playlists based on user like/dislike ratings may not intelligently adjust the playlist when the user's health or context changes.

The inventors have recognized and appreciated that a better way to select media content items involves tracking the user's media preference and consumption patterns in different contexts, and objectively evaluating the effects of media content items on aspects of the user's health (e.g., mood, physiology and behavior). Such characterization of media-health interactions can enable users to make informed choices of media content suitable to specific contexts (e.g., activities and environments), and suitable for eliciting a target change in the user's health.

Conventional monitoring systems do not continuously monitor and synthesize data on the media consumed by users in the absence of defined, user-conscious activities, or during the user's everyday living when media content may be passively consumed by the user as a secondary activity and without special attention to its selection and properties (for instance, when the user is daydreaming, or getting ready for work, or in commercial places such as shops and restaurants). Conventional monitoring systems do not properly interpret the effect of media consumption on the user in a broader context. Conventional monitoring systems do not assess and predict how a user would respond to the same media content item if it were rendered in multiple different contexts. The inventors have recognized and appreciated that intermittent snapshots of the user's health and media preference generally do not provide sufficient data to explore statistically meaningful associations between the media consumed by a user (or the features thereof) and the effects of that consumption on the user.

There is a need for computational systems that continuously track a user's health, context, and media consumption, integrate the user's health data, context data, and media data, analyze the integrated data to provide a detailed understanding of the user's media consumption and its effect on his/her health, and recommend media content items to the user based on objective measurements of the effects of consuming media content items on the user's health. There is a related need for systems and methods that facilitate continuous monitoring and mapping of a user's media consumption to various health data across different environmental contexts and through various activities of daily living. Such mapping can enable a user to derive insight into his/her media consumption in a comprehensive way and as it relates to his/her health and contexts. Such mapping can enable users to objectively categorize their media preferences in different contexts and to better select media suitable for specific situations and activities. Such systems can further provide personalized recommendations to the user on the appropriate media content to consume to maintain or achieve a desired health effect.

While it was previously infeasible to continuously monitor a user's health, there exist today many sensors and devices that monitor aspects of the health of individuals unobtrusively, at high resolution, and in everyday settings. One such example is the Fitbit® device that passively and continuously measures a user's steps, caloric burn, sleep patterns and overall activity throughout the day. With increasing use of smart device based media streaming applications, it is also possible to obtain continuous data on the user's media stream in different contexts.

One or more embodiments described herein may have one or more advantages or benefits. In some embodiments, the user's health is monitored before, during, and after the user's consumption of a media content item, and any resulting change in user health due to the consumption of the media content item is measured, and the magnitude of this effect is determined. The inventors have recognized and appreciated that such monitoring facilitates proper and comprehensive characterization of the effect of the media on user health.

In some embodiments, the user's health and media consumption are monitored without requiring the user to manually provide significant amounts of input. The inventors have recognized and appreciated that reducing the amount of user input facilitates more widespread adoption and more pervasive use of the system, which may enhance the quality and accuracy of the system's analysis of interactions between media consumption and user health. The present disclosure describes, according to some embodiments, a computational platform and methods of aggregating data on a user's health, context and media consumption at high-resolution from multiple devices and software applications without any restrictions regarding the type of device, source software or physiological signal, and with or without direct user input, analyzing the aggregated data to discover associations between consumption of media content and user health and context, and providing personalized recommendations of media content items for the user's health.

The present disclosure provides, according to some embodiments, a system and methods of aggregating data on a user's media consumption and health in one or more contexts. The system may monitor the user's media consumption in different contexts and throughout his various daily activities. The system may continuously capture data on the user's media stream, and his health before, during and after media consumption. Media data may be captured from the user's media player, and health data may be acquired through direct user input or passively measured via one or more sensors that monitor the user's health. The system additionally may aggregate information on the user's context (e.g., his/her environment and the types of activities he/she is engaged in) through sensors or user input.

The present disclosure provides, according to some embodiments, a method for mapping a user's acquired media data to his health measurements (e.g., physiological, psychological and/or behavioral health measurements) and context, at the time the media was presented. Time synchronized data may be analyzed to evaluate meaningful relationships between media and the user's health. In one embodiment, these analyses are aimed at characterizing a user's personal media preferences during different activities and in various environments, and evaluating the effects of individual media content items and their constituent features on the user's health. In some embodiments, any identified relationships are incorporated into a personalized media-health-context profile for the user. In some embodiments, the system provides a biofeedback mechanism by which the user is suggested or presented media content items that are suitable to his current health and context or effective in driving him to a desired target condition based on his personalized media-health-context associations. In some embodiments the analyses identify and/or characterize patterns in media consumption that are associated with, or predictive of, specific health conditions (e.g., physiological, psychological, and/or behavioral health conditions) of an individual or a population. The “media biomarkers” described herein may include such media consumption patterns. In some embodiments, interactions between the media consumption data and health data may be presented to the user by mapping the data to visual or sensory presentations.

The media-health relationships that are identified by some embodiments of the system and methods, and determined to be similar in multiple users or in a large population may be used to generate metadata tags that indicate the predicted health effect of consuming a media content item. These tags may be applied to the classification and cataloguing of media content items and media content types according to their health effects, and to generate libraries of media content suitable for different health conditions.

The present disclosure provides, according to some embodiments, a personalized platform for a user to monitor and evaluate the consumption of media content and its effect in everyday living, and to select or receive media content that is suitable to his/her various activities and health. The platform may be implemented as a personalized media therapy tool that is self-prescribed, or administered by a medical professional. In some embodiments, the platform is implemented as a research platform (or “research tool”) to investigate the effects of media consumption on various aspects of health. Other applications may include platforms for stratifying populations based on their personal media preferences for targeted marketing of media content, as well as evaluative platforms that assess user responses to promotional and marketing media and their effect on purchasing, purchasing intent, and consumer behavior.

The present disclosure provides, according to some embodiments, a system and method of aggregating and analyzing a user's media consumption pattern in context of his/her health (e.g., physiology, psychology, behavior) and environment throughout his/her daily living, and categorizing media content based on its correlation with or effect on these parameters. Analyzing the user's media consumption pattern in context of the user's health may include predicting and/or visualizing interactions between the user's patterns of media consumption and the user's health. In some embodiments, the system provides biofeedback to the user, whereby selected media content is recommended or rendered to the user based on the user's measured health responses (e.g., physiological, psychological and/or behavioral responses) to the media content and on the user's media preferences, to test, maintain or achieve a desired change in the user's health status (e.g., physiological, psychological or activity status).

According to an aspect of the present disclosure, a method of aggregating and analyzing a user's health and media data is provided, including: receiving user health data, receiving data on the user's media consumption pattern, synchronizing the time series of the user health data to the time series of the media consumption pattern, and analyzing the synchronized data for relationships between user health and media consumption pattern.

These and other aspects can optionally include one or more of the following features. In some embodiments, receiving data includes aggregating data from any hardware or software application or database configured to measure, store or represent information related to health or media. In some embodiments, receiving health and media data includes aggregating data from direct user input. In some embodiments, receiving health and media data includes passive aggregation of data from one or more sensors or devices configured to detect health or media metrics. In some embodiments, receiving data includes querying an external or third-party database or server and receiving a response. In some embodiments, receiving data includes aggregating data from a suitable media player or media streaming software application. In some embodiments, receiving data includes aggregating data continuously or at discrete time intervals. In some embodiments, the method further includes receiving context data corresponding to the user's context.

In some embodiments, data on each health and media metric is aggregated independently or is aggregated as a combination of multiple metrics. In some embodiments, heath data includes one or more parameters related to user physical state, physiology, psychology, mood, activity, and/or behavior. In some embodiments, context data includes one or more parameters corresponding to the user's environment and/or other context. In some embodiments, psychology data includes information on mental states obtained from suitable sensors or from clinical assessment scales. In some embodiments, the mental states include fatigue, depression, stress, and/or anxiety. In some embodiments, health data includes information on overall wellbeing and/or physical state of the user obtained by direct input and/or from clinical assessment scales and reports. In some embodiments, mood data is obtained by direct user input on a mood-arousal grid or a visual or numeric rating scale. In some embodiments, physiology data includes vitals data obtained from sensors or clinical monitors. In some embodiments, vitals data includes heart rate, blood pressure, and/or breathing rate. In some embodiments, physiology data includes brain waves and/or brain activity signals measured by MRI and EEG monitors. In some embodiments, activity data is collected via a pedometer, accelerometer and/or gyroscope. In some embodiments, activity data includes steps, pace, gait, and/or overall movement level. In some embodiments, activity data includes information on the nature of an activity collected by direct user input or from sensors configured to auto-detect the activity. In some embodiments, the activity is sleeping, reading, or driving. In some embodiments, context includes geographic data. In some embodiments, the geographic data is obtained from a GPS receiver. In some embodiments, environment data includes weather data. In some embodiments, the weather data is obtained via direct user input or by a weather software application.

In some embodiments, the user's media consumption pattern includes data on the name, type, composition, and/or characteristics of media content consumed by the user, timing of the user's media consumption, frequency of the user's media consumption, and/or associated metadata. In some embodiments, the media content includes analog or digital information in any format. The format can be single or multi-dimensional, perceptible or imperceptible, real or virtual. In some embodiments, the format includes auditory, visual, haptic, and/or olfactory data. In some embodiments, the media content includes a piece of music or audio content. In some embodiments, characteristics of the music include compound acoustic features, low level acoustic features and patterns in individual or combinations of acoustic features.

In some embodiments, the data are synchronized by aligning time stamps of two or more data streams continuously or at discrete time intervals. In some embodiments, the timestamps of the two or more data streams are aligned at time intervals of one second.

In some embodiments, analyzing the synchronized data for relationships between user health and media consumption pattern includes performing a mathematical, computational or statistical operation to identify correlations and/or other relationships between health data and media data. In some embodiments, the relationships include short-term and long-term correlations between media data and health data. In some embodiments, the correlations include identification of media consumed concurrent with, or within a specified time interval preceding or after a health event.

According to another aspect of the present disclosure, a method of personalized classification of media content for health is provided, the method including: receiving user health data, receiving data on the user's media consumption pattern, synchronizing the time series of the user health data to the time series of the media consumption pattern, analyzing the synchronized data for relationships between user health and media consumption pattern, and constructing a user's personalized media-state profile describing relationships between media and user health.

In some embodiments, the media-state profile describes a user's past health response(s) to an individual media content item or groups of media content items. In some embodiments, the media-state profile describes a user's current or predicted health response to a media content item based on the user's past response to the same media content item or a similar media content item. In some embodiments, the media-state profile describes a user's past, current or predicted health response to a specific feature of media content, or to groups or patterns of features of media content. In some embodiments, the media-state profile describes a user's predicted health response to media content by comparing the user's media-state profile to profiles of other users or groups of users. In some embodiments, media-state profiles aggregated from groups of users are used to generate health tags for media content items describing one or more health effects of the media content items on the group or population.

According to another aspect of the present disclosure, a method of providing personalized therapy is provided, the method including: receiving data on current user state, receiving data on desired user state, referring to the user's personalized media-state profile to identify a media content item suitable to the current or desired user state, displaying the identified media content item on the user interface and/or rendering the media content item through a media player, and receiving data on a new user state.

In some embodiments, a desired user state is pre-specified by a user or caregiver or obtained by direct user or caregiver input. In some embodiments, a desired user state is obtained by referencing a prescribed personalized or standard clinical program. In some embodiments, a desired user state is automatically computed based on the user's stored or measured health profile.

According to another aspect of the present disclosure, a method for presenting sensory information regarding an interaction between media consumption of a user and health of the user is provided, the method including: obtaining health data regarding the health of the user, obtaining media data regarding the media consumption of the user, temporally synchronizing the health data and the media data, mapping at least a portion of the media data and at least a portion of the health data to values of one or more sensory parameters, wherein the portion of the media data and the portion of the health data are associated with a same time period in the time series, and presenting sensory information to the user, wherein the sensory information represents the values of the one or more sensory parameters.

In some embodiments, the sensory information includes visual information, auditory information, tactile information, olfactory information, and/or taste information. In some embodiments, mapping at least the portion of the media data and at least the portion of the health data to the values of one or more sensory parameters includes mapping at least the portion of the media data to a first visualization parameter and mapping at least the portion of the health data to a second visualization parameter. In some embodiments, the first visualization parameter includes a color, transparency, shape, rotation, and/or pixilation of a graphic, the second visualization parameter includes a color, transparency, shape, rotation, and/or pixilation of a graphic, and the first visualization parameter differs from the second visualization parameter. In some embodiments, the health data includes values of a first health parameter for the user, the one or more sensory parameters include a first sensory parameter, and mapping at least the portion of the media data and at least the portion of the health data to the values of the one or more sensory parameters includes: generating correlation data regarding a correlation between the portion of the media data and the values of the first health parameter, and mapping the correlation data to the first sensory parameter.

According to another aspect of the present disclosure, a method for identifying a media biomarker associated with a health state is provided, the method comprising: obtaining health state data regarding a health state of a population, obtaining a media consumption signature associated with media consumption by the population, generating correlation data regarding one or more correlations between the health state data and the media consumption signature, identifying an association between the health state of the population and the media consumption signature based, at least in part, on the one or more correlations between the health state data and the media consumption signature, and in response to determining that a strength of the association between the health state and the media consumption signature exceeds a threshold strength, identifying at least a portion of the media consumption signature as a media biomarker associated with the health state.

In some embodiments, the population includes an individual person, plant, or animal, or a plurality of people, plants, or animals. In some embodiments, the plurality of people, plants, or animals have one or more characteristics in common. In some embodiments, the media consumption signature includes data relating to or derived from media consumption by the population and/or media consumption preferences of the population. In some embodiments, the method further includes using the media biomarker to diagnose the health state, to track the health state, to predict an effect of consuming media associated with the media biomarker on a health of the population, to generate a music playlist for modulating one or more health parameters of a member of the population, to generate a music playlist for consumption by a member of the population in conjunction with using a drug, and/or to provide biofeedback to a member of the population.

In some embodiments, analyzing the synchronized data for relationships between user health and media consumption pattern includes: determining a correlation value between the user health and the media consumption pattern, and comparing the correlation value to a threshold value. In some embodiments, analyzing the synchronized data for relationships between user health and media consumption pattern includes: determining a correlation value between the user health and the media consumption pattern, and comparing the correlation value to a threshold value. In some embodiments, determining that the strength of the association between the health state and the media consumption signature exceeds the threshold strength includes comparing a correlation value from at least one of the one or more correlations to a threshold value.

Further Motivation for and Advantages of Some Embodiments

Particular embodiments of the subject matter described in the present disclosure can be implemented to realize one or more of the following advantages.

Consumption of media content can have therapeutic effects on a person's health. For example, consumption of certain media content can help a person maintain a current health state, attain a target health state, or overcome a health condition. However, the health effects of consuming media content are generally not well-understood, and the health effects of consuming particular media content items may vary among different people. Thus, there is a need for systems and techniques for reliably predicting the effect on a person's health of consuming a particular media content item.

The inventors have recognized and appreciated that the relationship between media consumption and health can be better understood by analyzing how a person's health responds to consumption of portions of media content items that exhibit particular features (e.g., low-level audio features of the media content items, features relating to harmonic complexity of music, etc.). In some embodiments, the results of such analysis can be used to reliably predict the effect of consuming a particular media content item on the health of a person or a population. In some embodiments, the results of such analysis can be used to provide biofeedback to a person to help the person maintain a current health state or attain a target health state. In some embodiments, the results of such analysis can be used to prescribe consumption of one or more media content items as a therapy for a person who has a health condition or is undergoing a medical intervention for a health condition.

In addition, the inventors have recognized and appreciated that the media consumption signature(s) (e.g., amounts, rates, and/or patterns of media consumption) of a person or population can indicate that the person or population has certain health conditions. Thus, the media consumption signatures exhibited by a person or a population can be predictive biomarkers for health conditions in the person or population. For example, a pattern of greater than 70% frequency of listening to music in the acid rock genre with combined acoustic properties of tempo greater than or equal to 120 beats per minute (bpm), high entropy, and high percussion amplification may be indicative of depression in the listener.

Using conventional techniques for tracking media consumption and health in individuals or populations, it has not been possible to determine which media consumption signatures are reliable predictors of health conditions. However, some embodiments of the present disclosure can be used to determine which media consumption signatures are reliable predictors of health conditions, and to detect those media consumption signatures in individuals or populations. The presence of such media consumption signatures (“media biomarkers”) can be relied upon as predictive biomarkers in medical diagnostic processes. In other words, the presence of certain media biomarkers in a person (or population) can be used to assist in the diagnosis of the person's (or population's) health conditions.

Identifying and Exploiting Relationships Between Media Consumption and Health

Terms

As used herein, the terms ‘user’ and ‘listener’ include any individual that interacts with a system for identifying and/or exploiting relationships between media consumption and health (e.g., to track media consumption, listen to music rendered by the system, identify and/or visualize correlations between health data and media consumption data, etc.).

As used herein, the term ‘media’ includes any analog or digital information or data, in any single or multi-dimensional, perceptible or imperceptible, real or virtual, single or combinatorial auditory, visual, haptic, taste-based, or olfactory format.

As used herein, ‘health data’ may include values of health parameters related to a population's health. Health parameters may include, without limitation, any physical parameter, physiological parameter, psychological parameter, emotional parameter, cognitive parameter, behavioral parameter, well-being parameter, clinical parameter, mood parameter, activity status, and/or other parameter that relates to any aspect of a population's health or well-being. In some embodiments, health data may include patterns relating to the population's health (e.g., patterns in the values and/or arrangement of health parameters over time). In some embodiments, health data may include individual values of an individual health parameter, individual values of multiple health parameters, combined values of an individual health parameter, combined values of multiple health parameters, patterns of individual health parameters, and/or patterns of combined health parameters.

Non-limiting examples of health parameters include heart rate, heart rate variability, blood pressure, respiration rate, galvanic skin response, emotion, mood, valence, EEG signal, EKG response, pulse, activity, blood glucose, etc. Some health parameters (e.g., “complex” health parameters) may be determined based on other health parameters. Examples of complex health parameters include, without limitation, level of depression, stress, diabetes, ADHD status, overall health/wellness status, genomic profile, metabolomic profile, microbiome profile, neurological profile, etc.

As used herein, ‘media data’ may include a type of media (e.g., audible media, visual media, audiovisual media, videos, images, text, music, speech, ambient acoustics, etc.), and/or attributes of the media. The attributes of music media may include data that identifies the music (e.g., the songwriter, performance artist, song title, album title), the genre or type of the music, instruments used to produce the music, acoustic properties (e.g., beats per minute, pitch, key, volume, etc.), delivery mechanism (e.g., live performance, playback of a recording), rhythm, beat, etc. The attributes of text media may include the author, genre, topic, delivery mechanism (e.g., magazine, newspaper, book, internet), etc. The attributes of video may include the genre, actors, director, producer, title, etc. The attributes of image media may include the image type (e.g., photograph, painting, drawing, etc.), artist (e.g., photographer, painter), subject (e.g., people, places, or things depicted in the image; concepts conveyed by the image), etc.

As used herein, ‘media consumption data’ may include data describing media consumption by a user or population (e.g., data describing a history of a media consumption or a pattern of media consumption, media data describing the consumed media, etc.). A pattern of media consumption may include changes in the amount or rate of media consumption over time, etc.

As used herein, the terms ‘music’ and ‘song’ may include any segment of audio content of any length and composition (e.g., a song, a music composition, an instrumental piece, a sequence of tones or natural or artificial sounds, or specific features or elements of the above).

As used herein, the term ‘synchronization’ includes all computational, mathematical and statistical operations performed to match the time series of media consumption data to the time series of health data and/or contextual data to align the timestamps of concurrent events in the individual data streams.

As used herein, the term ‘mapping’ includes any computational, mathematical and/or statistical operations performed to identify and evaluate any associations (e.g., correlations or other relationships) between parameters in the media consumption, health, and contextual data streams.

As used herein, a “media biomarker” may include media consumption data (e.g., media consumption signatures, including but not limited to media consumption patterns) associated with, or predictive of, specific health states (e.g., conditions) of an individual or a population. In some embodiments, the media consumption pattern associated with or predictive of a particular health state may be a pattern indicating a change in media consumption of a particular magnitude (e.g., an increase or decrease of 10% or more in the frequency of consuming media content), a change in media consumption in a particular direction (e.g., an increase or decrease in the amount of media content consumed), or any other change in media consumption.

A media biomarker may include data indicating the strength of the association (e.g., correlation) between the media consumption signature or pattern and the corresponding health state. A media biomarker may be predictive or diagnostic. Predictive biomarkers may indicate that media consumption consistent with the biomarker's signature is predicted to drive the user's health state toward the health state corresponding to the biomarker. Diagnostic biomarkers may indicate that the presence of the biomarker's signature in the user's media consumption data is predictive of the user being in the health state (e.g., having the health condition) corresponding to the biomarker. A media biomarker may specify, without limitation, a type of media (e.g., audible media, visual media, audiovisual media, videos, images, text, music, speech, ambient acoustics, etc.) features of the media, and/or attributes of the media. In some embodiments, a media biomarker may indicate whether a health parameter value precedes the media biomarker or whether the health parameter value increases, decreases, or stays the same during or after consumption of media.

The term “sensory information,” as used herein, may include, without limitation, information that can be sensed by sight (visual information), sound (auditory information), touch (tactile information), smell (olfactory information), taste (taste information), and/or any combination thereof (e.g., audiovisual information).

As used herein, “consuming media” may include, without limitation, any act or state whereby an individual or population senses or perceives media content (e.g., reading, listening, viewing, or otherwise sensing or perceiving the media content).

As used herein, ‘features’ of media content may include characteristics or parameters of music (‘music features’), of audio content (‘audio features’), of image content (‘image features’), of video content (‘video features’), of text content (‘text features’), of speech content (‘speech features’), and/or any other suitable characteristics or parameters of media content.

Music features may include, for example, features related to rhythmic timing (e.g., tempo, beat, beats per minute, tatum, rhythm), features related to sound quality (e.g., timbre, pitch, key, mode, volume, loudness), features related to harmonic complexity (e.g., key, mode, pitch), features related to musical preference (e.g., genre, style, artist, artist location, artist familiarity), or features related to subject perception of the music (e.g., hotness, danceability, energy, liveness, speechiness, acousticness, valence, mood). In some embodiments, danceability may be determined based at least in part on tempo, rhythm stability, beat strength, and/or regularity of the music. In some embodiments, energy represents the intensity or activity of the music, and may be determined based at least in part on dynamic range, loudness, timbre, onset rate, and/or general entropy of the music. In some embodiments, liveness represents the presence of an audience in the music. In some embodiments, speechiness represents the presence of spoken words in the music. In some embodiments, acousticness represents the extent to which the music was created using acoustic (rather than electronic) techniques. In some embodiments, valence represent the positivity (e.g., happiness, cheerfulness, or euphoria) conveyed by the music.

Music features may include, for example, simple features relating to fundamental structural elements of music (e.g., key, tempo, pitch, etc.) or complex features that result from combining two or more simple features (e.g., groove, danceability, energy, etc.).

Music features may include, for example, low-level audio features. In some embodiments, low-level audio features include standardized low-level features described in the MPEG-7 standard (MPEG-7 Multimedia Content Description Interface Parts 1-14, ISO/IEC 15938, which is hereby incorporated by reference to the maximum extent permitted by applicable law). In some embodiments, low-level audio features include features directly extracted from a digitized audio signal (e.g., from independently processed frames of a digitized audio signal). Some non-limiting examples of low-level audio features include Mel-Frequency Cepstral Coefficients (MFCC), Audio Spectrum Envelope (ASE), Audio Spectrum Flatness (ASF), Linear Predictive Coding Coefficients, Zero Crossing Rate (ZCR), Audio Spectrum Centroid (ASC), Audio Spectrum Spread (ASS), spectral centroid, spectral rolloff, and/or spectral flux.

Music features may include, for example, “compound” or “high-level” features. In some embodiments, compound features include features that can be directly perceived by humans. In some embodiments, a compound audio feature includes a combination of one or more low-level audio features, one or more sound-quality audio features, and/or one or more harmonic complexity audio features. Some non-limiting examples of compound features include tempo, timbre, rhythm, structure, pitch, beats per minute, and melody.

Music features may include, for example, acoustic features. Some non-limiting examples of acoustic features are described in U.S. Pat. No. 8,583,615, which is hereby incorporated by reference to the maximum extent permitted by applicable law.

Video features may include, for example, color, brightness, motion, and director.

Image features may include, for example, color, brightness, and author (e.g., photographer or painter).

Text features may include, for example, tone, voice, genre, and author.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of,” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Systems and Techniques

In some embodiments, a system for identifying and/or exploiting relationships between media consumption and health comprises a Health Module, Media Module, Synchronization Module, Analysis Engine, and User Interface, which are described in detail below. It is to be noted that groupings of alternative elements of some embodiments of the invention disclosed herein are not to be construed as limitations. Each element can be implemented independently and can be referred to and claimed individually, or in any combination with other elements or groups of elements described herein.

Some embodiments can be practiced with any computer system configuration including desktops, mobile computing devices (e.g., the Amazon Kindle®, Apple iPad® and the Windows Surface™ tablets), smart mobile communications devices (e.g., the Apple iPhone®), smart watches (e.g., the Apple iWatch® and the Samsung smart watch), portable music players (e.g., the Apple iPod® and the ZUNE® music player), wireless music systems (e.g., the Sonos® smarthome system), multiprocessor-based or programmable consumer electronics, network PCs, minicomputers, mainframe computers and the like. Implementations may also be practiced in distributed and cloud computing environments (e.g. Amazon EC3), where tasks are performed by remote processing devices that are linked through a communications network. In a distributed computing environment, system modules may be located in both local and remote computing devices.

Health Module

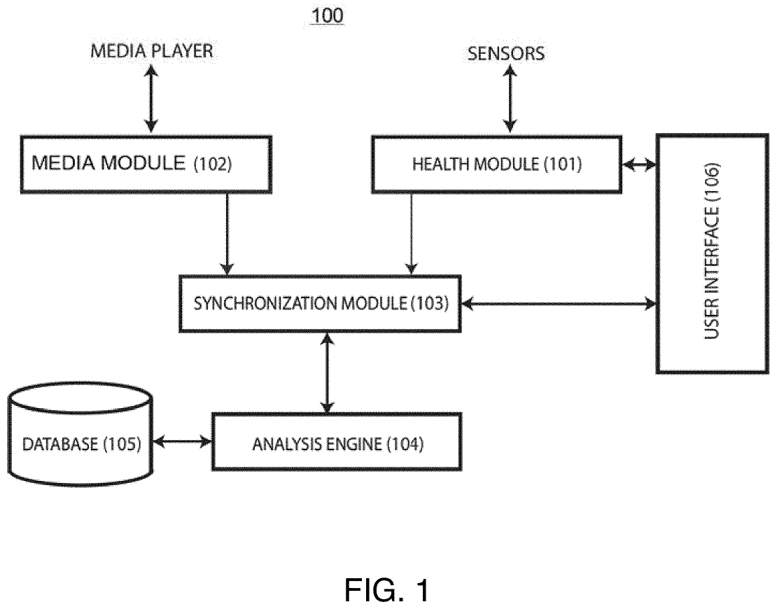

Referring to FIG. 1, some embodiments of a system 100 for identifying and/or exploiting interactions between media consumption and health include a Health Module 101 that aggregates and processes a user's health data. The user's health data may be obtained by embodiments of the system using, for example, any of various suitable methods described below.

The health module 101 can obtain the user's health data via active user input (e.g., in response to an invitation to the user to input his/her health information via the User Interface 106). The system 100 may invite user input via simple notifications delivered to the user's smart device or computer. The notifications may include, for example, text, audio data, visual data or haptic data. In one embodiment, the active input is obtained from the user by answering questions related to his/her health. Questions may be presented in the form of surveys, standard clinical questionnaires, mood or activity scales, or in any numerical, pictorial or graphical form suitable for conveying a range of health conditions to the user and capturing the user's input. Questionnaires and scales can be specific to health conditions, for example, anxiety (e.g. the Hamilton Anxiety Scale, Generalized Anxiety Disorder 7 questionnaire, Symptom Checklist, Zung Self-Rating Anxiety Scale etc.), depression (e.g. the Patient Health Questionnaire, Major Depression Inventory, Geriatric Depression Scale, Beck Depression Inventory etc.), fatigue (e.g. the Brief Fatigue Inventory, The Profile of Mood States Fatigue/Inertia Subscale, Rhoten Fatigue Scale, Fatigue Impact Scale, Multidimensional Fatigue Symptom Inventory etc.), pain (e.g. the Visual Analogue Scale, Verbal Rating Scale, Numerical/numeric Rating Scale), sleep disorders (e.g. the Pittsburgh Sleep Quality Index, Epworth Sleepiness Scale), etc. Alternately, questionnaires and scales may not be specific to health conditions, but may be generally related to health (e.g., the Quality of Well Being Questionnaire, Self-perceived Quality-of-life scale, the Sickness Impact Profile, the valence-arousal grid for general mood, etc.). In some embodiments, many types of questions, scales, and forms of the type used in clinical, educational, and epidemiological research are used. In some embodiments, the user may enter health and/or contextual data that he/she may have obtained through an unconnected device or through rudimentary means. For example, a user may enter his weight measured on an analogue weighing scale, or his daily steps that have been manually recorded in a diary or his blood glucose levels measured on a glucose monitor. In some embodiments, the health module may acquire health data of a user through input by a caregiver or informant, for example a doctor, nurse, family-member, teacher, friend, etc. Information can be acquired from a single caregiver or be acquired and consolidated from multiple informants. The informant may enter assessments of and observations on a user's health state (e.g., observations regarding his/her mood, behavior, symptom severity, etc.).

In some specific embodiments where user health information is actively entered, the system may gamify the user notification and data input process to ensure that it is motivational and engaging to the user. For example, the system 100 may provide the user a score or rewards that accrue as the user enters his/her health information in a timely and complete manner. In some embodiments the system may present to the user a puzzle or a quiz that requires the user to input his/her health information.

In some embodiments, the Health Module can obtain the user's health data passively from one or more sensors that measure, for example, a physiological, physical or activity parameter of the user. Sensors can be externally attached to the body, can be contact-free from the body and operate remotely, or can be internally implanted in any suitable location of the body. Such sensors may sense either independently, or some combination of the user's physiological data and vitals, including but not limited to the heart rate, cardiac rhythm, blood pressure, pulse rate, body temperature, blood pressure, EKG data, EEG data, skin conductance, hydration levels, blood flow, blood gas content, breathing rate, lung volume, and blood or tissue metabolite levels. Some sensors may sense physical activity data, for example, overall movement levels, step count, stride distance and symmetry, gait, number of jumps, jump height, falls, distance traveled, speed, step impact force data, other accelerometer and gyroscope based data, calorie expenditure, sleep movements and quality, etc. In some embodiments, the user's physical activity data is obtained directly from athletic training machines and gym equipment, for example, treadmills, spinning machines, elliptical training machines, stationary bicycles, stair climbing machines, cross-country ski stimulating machines, weight lifting machines and rowing machines. In some embodiments, health data can be transcribed from medical and health records (e.g., electronic medical records, personal health records, lab reports, etc.) or be obtained from virtual avatars that provide a representation of the health condition of a user. In some embodiments, the system 100 uses measures and systems used in physical, neurological, physiological, genomic, proteomic, metabolomic, microbial and/or associated human research or clinical practice to obtain a user's health data.

The health module may capture data actively or passively on the user's context. Context may include any parameter relating to the user's current environment. Context can include information regarding the type(s) of activities the user is currently engaged in, for example, exercising at the gym, sleeping, or driving to work or whether he/she is alone or with company. In some embodiments, context includes temporal data, for example, the time of day, month or year, or the current position of an individual's infradian, circadian or ultradian biological rhythms. Context can include meteorological data, for example, current weather, temperature, humidity, precipitation, barometric pressure or season. In some embodiments, context includes data related to the user's current geographical location, for example, the GPS location, type of terrain, altitude, traffic patterns, ongoing or recent events around the user's current location, or other local information. Context can comprise historical data on the user's past associations with any parameter relating to his/her current environment. For example, content may include information on the user's past mental states while driving in a specific location on previous occasions. In some embodiments, any parameter related to the user's current environment or behavior that may influence his/her health or media preference is suitable to be measured as context. In some embodiments, context includes information on the user's target or desired health state or outcome.

In some embodiments, the health module is always enabled to aggregate health data on an ongoing, continual basis. In some embodiments, the user may disable the health module and enable it at specific time intervals (e.g., every few seconds, or minutes or days). The interval of data acquisition may either be pre-determined or fixed by some embodiments, or it can be adjusted by the user by input via the user interface. In some embodiments, the system may recommend to the user data-acquisition intervals (e.g., optimal data-acquisition intervals) for a specific sensor or health parameter. Additionally, in cases where data is acquired on multiple health parameters or from multiple sources, the health module may sync each parameter or device at independent time intervals. For example, the system may continuously upload data on the heart rate or blood pressure of a user, however it may upload blood glucose readings only once a day or once every couple of days. In some embodiments, health data may be aggregated only during periods of active media consumption. Alternately, in other embodiments, health data may be acquired continuously during periods of media consumption as well as media inactivity.

The health module conveys the aggregated health data (and, optionally, contextual data) to the Synchronization Module (103) where the health data (and, optionally, contextual data) are mapped to the user's media consumption data. In some embodiments, the health module may directly transmit the health (and contextual) data in an unprocessed, raw format in which it is acquired from the user interface and sensors. In other cases, the health module first processes the data. Data processing may involve operations including normalization, manipulation of data types and formats, synchronization of multiple data streams, and/or analysis of the acquired health (and contextual) parameters to infer a user health condition. The health module may communicate the health (and contextual measurements), along with the timestamps indicating the times at which these measurements were acquired, to the synchronization module as a unified signal or as multiple, independent data streams.

Media Module

The Media Module 102 functions to collect data on the user's media consumption. The media module may interact with any media player that presents media to the user. Suitable media players may include traditional hardware players (e.g., CD-ROM and cassette players) or software applications (e.g., on smart devices) that stream media stored on the local device or from a remote server (e.g., an internet radio server or video server). Examples of such applications include Apple's iTunes player, Windows Media Player, the Pandora, Spotify or Beats applications, or the Netflix service. The media module may obtain from these media players data on the sequence of media content items (e.g., music, songs, videos, etc.) presented to the user and the times at which they were presented. In some embodiments, the media module acquires metadata corresponding to the media content items, for example, metadata indicating (for a song) the name of the song, artist or album, or genre. In some embodiments, metadata includes information on the features of the media content item. In some embodiments, the media module acquires metadata directly from the interfacing media player. In some embodiments the media module queries external, third-party databases (e.g., the Echonest database) to acquire information on the media content item's features. In some embodiments, the media module acquires the computer-readable representation of each media content item rendered to the user, and presents the representation of the item to the Analysis Engine (104) for analysis of its features. Features may be obtained or analyzed for the entire media content item or for a fraction of the content item of any length or time period. The media module may generate one or more data streams of the acquired media data including the name of the media content item, associated metadata and timestamps to convey to the synchronization module. The media module may generate a signal to the interfacing media player to play selected media content items.

In addition to music or songs, other media content may be suitable to some embodiments of the system and methods disclosed herein. Suitable media items may include audiovisual data (e.g., videos), virtual 3D environments, speech and audio clips, images, photos, or text data. In these implementations, the media module can interface with media players, hardware devices or software applications that are suitable for rendering the above media (for example, video cassette players, gaming environments and consoles, ebook readers, internet webpages, etc.). The media module may acquire and process metadata related to such media content items, including the length or size of the media content item and its associated features. Additionally, environmental sounds and ambient noise acquired directly by the media module or indirectly from devices and applications interfacing with the media module are also suitable for processing by some embodiments of system 100.

Synchronization Module

The Synchronization Module (103) receives data from the health and media modules and functions to map the health data (and, optionally, contextual data) to the media data. Mapping may be performed according to one or more suitable techniques, and by a variety of software tools suitable for synchronization of time-stamped data streams. See, for example, U.S. Pat. No. 7,765,315 and Ojeda, A. et al., Front. Hum. Neurosci., 2014, 8:(121): 1-9, each of which is incorporated herein by reference to the maximum extent permitted by applicable law. As an example, a data stream may be directly synchronized to another data stream, or each data stream may first be independently aligned to a common point of reference such as a reference clock and then synchronized to other data streams that have been similarly aligned to the common reference. Independent data streams may be synchronized in entirety, or may be first divided into fragments of any length that are then aligned together in any combination. Data streams may be synchronized in various time increments, for example, their timestamps may be aligned at intervals of milliseconds, or seconds, or minutes. In some embodiments individual data streams may be synchronized in phase (e.g., the nth timestamp in one data stream aligned to the nth time stamp in another data stream). In other embodiments data streams may be phase shifted prior to alignment, for instance the nth timestamp in one data stream may be aligned to the n−1, n+1, or n−2 time stamp in another data stream.

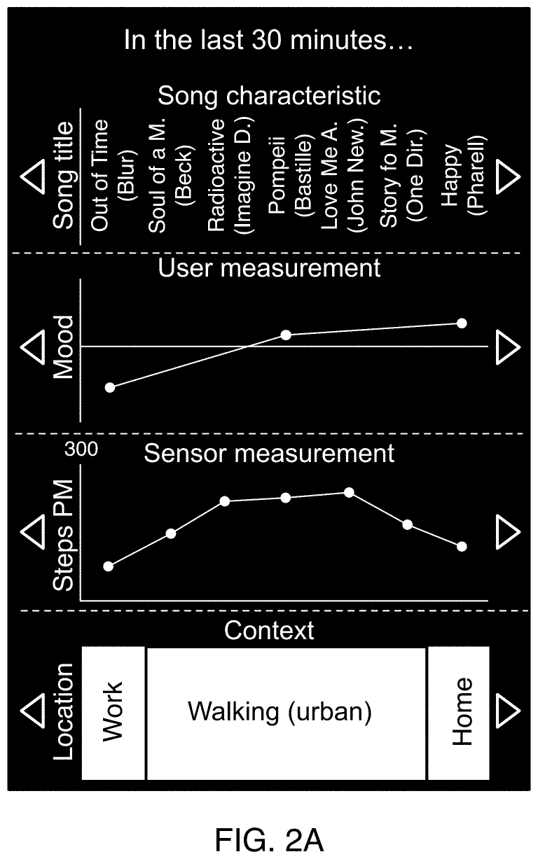

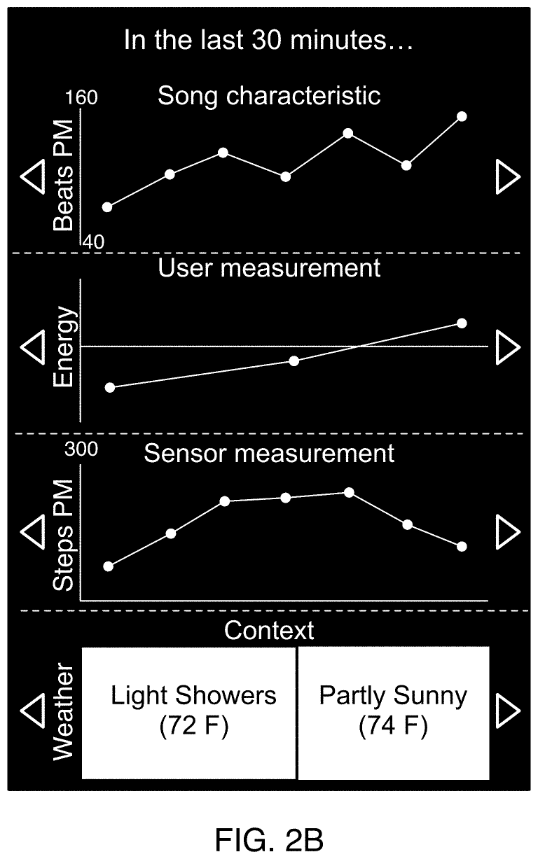

In some embodiments, the synchronization module constructs representations (e.g., graphical representations) of the synchronized data streams for presentation (e.g., visualization), and to allow users to explore relationships between their media consumption, and health and/or context. In a simple representation, shown in FIG. 2A, graphs may display a time series of songs the user consumed during an activity or during a specified time interval, and the synchronized time series of the user's activity level (e.g. number of steps), psychological parameters (e.g. mood), physiological parameters and context parameters (e.g. at home or walking) during that period. The synchronization module may additionally process and display different parameters related to the user's health, context and media data. For example, as shown in FIG. 2B, the synchronization module may graph the time series of the user's energy level inferred from his/her mood data, aligned with various metadata relating to the songs (such as the beats-per-minute or pitch) consumed by the user during a certain period. In some embodiments, the synchronization module displays these graphs to the user via the user interface. Graphs can be presented in an interactive fashion, allowing the user to select, filter and modify the data streams and their related parameters that are synchronized and graphically displayed. The synchronization module may actively communicate with the analysis engine for processing the health and music consumption data into alternate formats for display as selected by a user.

Analysis Engine

The Analysis Engine (104) receives synchronized health and music consumption data streams from the synchronization module and performs analyses to identify relationships between user media consumption and health (and, optionally, context). The analysis engine may implement mathematical operations to identify correlations between the frequency of consumption of individual media contents items or groups of media content items and user context to identify the user's media preferences during different activities and in different environments. In some embodiments, the system may measure changes in user health (e.g., mood, physiology, and behavior) throughout a period associated with the user's media consumption, including before, during and/or after consumption of each media item to determine the effect of the rendered item on the user's health. It is appreciated that the engine may identify short-term and/or long-term associations between user health, context, the duration of media consumption, the user's media preference, a type of media, a specific media content item, and/or features of media content items. In some embodiments, a short-term association exists when the health effects of consuming a media content item are detectable just prior to the user consuming the media item (e.g., an anticipatory response), immediately detectable upon the user consuming the media item, or detectable within seconds, minutes, or hours (e.g., up to approximately eight hours) after the user consumes the media item. In some embodiments, a long-term association exists when the health effects of consuming a media content item are detectable at least a specified time period (e.g., approximately eight hours) after the user consumes the media item. In some embodiments, an effect on the user's health is “detectable” if the corresponding health parameters can be detected using specified health sensors or medical tests. These associations may inform a personalized music-health-context profile of the user and may be incorporated into a predictive model that describes how a user responds to individual or groups of media content items or to features related to the media contents item(s) in various conditions. The personalized user profile and predictive model may be stored in a reference database (105), and in some embodiments may be engaged by the biofeedback module to provide suitable media content recommendations to the user.

To construct the user profile and predictive model, the analysis engine may implement one or more data-mining, feature selection, pattern recognition, signal processing, machine learning and other mathematical, computational or statistical approaches known in the art for analysis of large datasets. Some examples of such approaches are described in Hastie, T. et al., The elements of statistical learning, Vol. 2, No 1, 2009, Springer: New York; Bishop, C. M., Pattern recognition and machine learning, Vol. 1, 2006, Springer: New York; Duda, R. O. et al., Pattern classification, 2012, John Wiley & Sons Keppel, G., Design and analysis: A researcher's handbook, 1991, Prentice-Hall; and Maxwell, S. E., & Delaney, H. D., Designing experiments and analyzing data: A model comparison perspective Vol. 1, 2004, Psychology Press. Mathematical and statistical operations including but not limited to factor analysis, principle component analysis, linear discriminate analysis, multiclass logistic regression, and nearest-neighbor approximations in high-dimensional space can be employed by the analysis engine to identify correlations and relationships between media types or individual media content items and states or events in the corresponding health data and contextual data (for example to identify songs that correlate with an increase in heart rate or with sunny weather). The analysis engine may identify correlations between, for example, (1) concurrent health states and media consumption events or patterns, for instance the songs a user listened to at the peak of a health event, or (2) time-separated health and media content events, for instance the songs a listener consumed at some time interval before or after the peak of the health event. The analysis engine may implement factor analysis and/or linear regression methods to quantify the magnitude of the changes in user health observed on presentation of a media content type or item, and to predict future changes upon media presentation. Classical statistical tests (e.g., t-tests, ANOVA, ANCOVA and regression) may be implemented to characterize the statistical significance of these changes in user health.

In some embodiments, the analysis engine may be pre-seeded with default predictive models that have been developed based on relationships characterized in previous datasets from early platform adopters, testers and other users. These models can be personalized for a new user and trained on an ongoing basis when new data from the user becomes available. The analysis engine can also generate and test hypotheses of how individual media content items or types of media content can affect the user in previously untested contexts, as well as the effect(s) of untested media content items or types on the user.

The analysis engine can aggregate data from multiple users to evaluate if the statistically significant media-consumption/health relationships identified for a user are generalizable across sub-groups or groups of similar users or in a larger population. Relationships that have broad applicability across different user contexts and populations may be utilized in generating metadata attachments for media content items that characterize the observed or predicted health effect of consuming the media content item. For example, if consuming a particular media content item is determined to reduce heart rate by 10 beats per minute in all users it can be health tagged as ‘effective in cardiovascular regulation’. As another example, if consuming a media content item is determined to reduce pain following surgery in children, it can be health tagged as ‘effective for pediatric post-operative pain’. The analysis engine can utilize the metadata tags to categorize and catalogue media content items and types according to their health effects, and to generate libraries of media content suitable for treating various health conditions or encouraging various health states. A media content item may have one or more health tags and may be categorized and cross referenced in one or more media libraries depending on whether the item elicits a single or multiple health effects. Media libraries may be stored in a common reference database and in some embodiments may be accessed by the biofeedback module to select or recommend media content suitable to a user (e.g., for maintaining or changing the user's health state).

The analysis engine may implement media content analysis using digital signal processing (DSP) and information retrieval techniques to characterize the features of individual media content items, or to infer common features in a collection of media content items that constitute a common preference group for a user or elicit a same health effect or similar health effects in a user or a group of users. For example, the analysis engine may implement music content analysis using DSP and music information retrieval techniques to characterize the features of individual songs, or to infer common features in a collection of songs. Audio content analysis may involve using any type of audio content features to classify audio data. The analysis engine may identify patterns (for example, a Fibonacci sequence) in the arrangement of individual features or combinations of features in a specific media item or group of items. Audio content analysis may enable the system to identify key acoustic features that mediate the observed health effect of a music item or a type of music. For example, the system may discover that all songs effective in reducing a user's heart rate have a similar tempo of ⁻60 beats per minute. Audio content analysis may therefore be employed to characterize untested media content, and to generate hypotheses regarding the content's potential health effects on the user. As an example, if a new song that has not been previously monitored by the system is determined to have a tempo of 60 beats per minute, some embodiments of the system can generate and test the hypothesis that this song will be effective in modulating a user's heart rate based on its shared acoustic properties with music that is known to regulate cardiovascular parameters. Characterization of expected health effects obtained by comparison of acoustic signatures of untested and tested media content may utilized by the system in generating health metadata tags for cataloguing of new music content.

It is appreciated that in addition to music content, the analysis engine can process and catalogue other types of media content delivered via the media module. For example, the analysis engine can process and characterize audiovisual, speech and text files (e.g., videos, audiobooks, web clippings, etc.) to evaluate meaningful correlations between the consumption of these media items and user health (e.g., in different contexts) and to assess relevant effects that these media have on user health (e.g., physiology and behavior). In some embodiments, the analysis engine may process and catalogue effects of environmental sounds and ambient noise on user health.

In some embodiments, the analysis engine identifies a media biomarker associated with a health state of a population. The population may include an individual person (in which case the media biomarker is a personalized media biomarker) or a group of people (in which case the media biomarker is a group or population media biomarker). In some embodiments, the population includes a group of people who have one or more characteristics in common (e.g., demographic characteristics, area of residence, clinical condition, etc.). In some embodiments, the population includes one or more plants or animals.

A media consumption signature may include one or more media consumption characteristics of a population. In some embodiments, media consumption characteristics include the amount of media consumed by the population (e.g., frequency and/or duration of media consumption), patterns of the population's media consumption, and/or one or more media consumption preferences of the population. Media consumption characteristics of a population may be determined based on media consumption data associated with a population. Media consumption data associated with a population may be obtained from one or more media modules 102 that collect data related to a population's consumption of media. In some embodiments, media consumption signatures may be identified based on patterns in media consumption data and/or correlations between such patterns and health states.

Media consumption characteristics may be classified based on attributes of the consumed media, including, without limitation, the type of media (e.g., audible media, visual media, audiovisual media, videos, images, text, music, ambient acoustics, etc.). Some attributes of various types of media content are described above. Other media content attributes are possible. In some embodiments, media consumption characteristics may be classified based on features of the consumed media. Some features of various media types are described above. Other media content features are possible. In some embodiments, media consumption characteristics may include data indicating whether a health parameter value precedes a media consumption signature or whether the health parameter value increases, decreases, or stays the same during or after consumption of media.

Media consumption preferences may include information relating to the population's preference for consuming or not consuming various types of media. In some embodiments, the population's media consumption preferences may be expressed as binary values (preferred or not preferred). In some embodiments, the population's media consumption preferences may include a degree of preference for consuming or not consuming various types of media. In some embodiments, the population's media consumption preference data may include data indicating changes or patterns in the population's media consumption preferences over time.

The analysis engine may identify and/or measure one or more health states (e.g., health conditions) of a population based, at least in part, on analysis of health data obtained by health module 101 (e.g., health data of the population). A health state may include, for example, any physical, physiological, psychological, emotional, cognitive, behavioral, mood, or clinical condition of the user, or any condition of the user relating to user activity or well-being. Other types of health conditions are possible. The population's health state(s) may be identified based on values of individual health parameters or combination(s) of health parameters included in the health data, and/or based on associations between health parameters included in the health data. Such associations may be analyzed or measured over any suitable time period, including short time periods (e.g., time periods on the order of seconds, minutes, or up to approximately eight hours) and/or long time periods (e.g., time periods longer than approximately eight hours or on the order of days, weeks, months, years, or decades). As an example, the analysis engine may identify that a peak in the user's electrodermal activity indicated by a value equal to or greater than five times the median daily value of electrodermal activity over a ten minute period is associated with a health state identified as ‘anxious’. As another example, the analysis engine may characterize that a synchronous 10% decrease in heart rate from baseline, a switch to delta-waves in EEG activity, and one degree fall in body temperature is indicative of a health state identified as ‘deep sleep’. In another example, the analysis engine may characterize that a sustained 3-fold increase in heart rate and blood pressure over baseline values, combined with blood glucose levels between 150 mg/dL-200 mg/dL over a one month period is indicative of a clinical health state identified as ‘pre-diabetic’. Health states may be personalized (e.g., specific to a user), group-specific (e.g., specific to a group of users), or universal (e.g., within a specified population).

To identify the health state(s) of the population, the analysis engine may apply any suitable processing technique to the health data (e.g., independent of the media consumption data). To identify the population's media biomarker(s), the analysis engine may apply any suitable processing technique to the health data and the media consumption data. Suitable processing techniques for identifying health states and/or media biomarkers may include, without limitation, mathematical, statistical, computational, and/or deep learning and machine learning techniques. In some embodiments, techniques applied to identify the health state(s) and/or media biomarker(s) may include data sampling (e.g., optimal data sampling) (e.g. using Nyquist frequency based sampling, compressed sampling, sparse sampling, etc.); pre-processing of health and/or media consumption data for correlation analyses and/or pattern recognition analyses (e.g. normalizing raw values of health and/or media consumption parameters based on a pre-determined range, baseline, maximum, mean, median, or rolling sample); filtering anomalies from the data (e.g., separating true signal from noise in data using methods such as dimensionality reduction via Principal Component Analysis); determining the correlation between data points (e.g., how data points or certain types of data support/negate other data points or types of data, using methods such as unsupervised learning by K-means, spherical K-means, spectral clustering, non-negative matrix factorization, Gaussian Mixture Model, etc.); identifying true signals in data (e.g., using methods such as feature selection and feature engineering, Markov models and support vector machines); performing covariate analysis on multiple variables (e.g., health parameters) to identify/measure a unified health state (e.g. using logistic regression, multilayer perceptron); pattern recognition and pattern exception recognition analysis for identifying correlations and patterns between different health data obtained by the health module to identify health states; identifying correlations and patterns between health data and media consumption data; identifying causation between media consumption data and health data; etc.

A media biomarker may be identified by identifying an association between a media consumption signature of a population and a health state of the population. In some embodiments, to identify associations between media consumption signatures and health states, the analysis engine generates correlation data regarding one or more correlations between the health state data and the media consumption signatures. To generate correlation data, the analysis engine may identify and/or measure correlations or correlated patterns between health states and media consumption signatures. In some embodiments, the analysis engine may determine and/or measure causation between health states and media consumption signatures. In some embodiments, the analysis engine may seed identification of media biomarkers for a population based on aggregate media biomarker data from other groups/populations.

The analysis engine may determine the strength of an association between a health state and a media consumption signature. In some embodiments, the analysis engine may measure the correlation between the health state and the media consumption signature, and compare the correlation value to a threshold value. If the correlation value exceeds the threshold value, the analysis engine may identify the media consumption signature (or a portion thereof) as a media biomarker associated with the health state. The correlation between the health state and the media consumption signature may be characterized using any suitable metric, including, without limitation, Pearson's correlation coefficient and/or a rank correlation coefficient (e.g., Spearman's rank correlation coefficient, Kendall tau rank correlation coefficient). The correlation between the health state and the media consumption signature may be determined using any suitable technique, including, without limitation, linear regression (e.g., least squares estimation, maximum-likelihood estimation, Bayesian linear regression, quantile regression, mixed models, principal component regression, least-angle regression, etc.), nonlinear regression, adaptive regression, curve-fitting, analysis of variance, etc. The statistical significance of the correlation between a health state and a media consumption signature may be determined using any suitable technique.

In some embodiments, media biomarkers may be used to diagnose health conditions. For example, in cases where the existence of a media biomarker is sufficiently correlated with the presence of a health condition (e.g., a disease, disorder, or chronic condition), the health condition may be diagnosed in an individual by detecting the media biomarker in the individual's media consumption data. Alternatively, diagnostic tests may be performed to confirm the suspected presence of the health condition when an individual exhibits a media biomarker (alone or in combination with known symptoms of the health condition or markers for the health condition).

In some embodiments, media biomarkers may be used to track the status of a health condition in a population. For example, in cases where different values, combinations, and/or patterns of a media biomarker's attributes are sufficiently correlated with different states or severities of a health condition, the state of the health condition may be tracked in a population by monitoring the values, combinations, and/or patterns of the media biomarker's attributes over a period of time. As another example, the presence or absence of a media biomarker in a population's media consumption data over time can be used to track prevalence of the corresponding health condition in the population over time. In some embodiments, a media biomarker may specify quantitative values, combinations, and/or patterns of attributes. In some embodiments, a media biomarker may specify qualitative values (e.g., “high” or “low”), combinations (e.g., a high value for one attribute and a low value for another attribute), and/or patterns of attributes. In some embodiments, a media biomarker may specify quantitative and/or qualitative values, combinations, and/or patterns of attributes.

In some embodiments, media biomarkers may be used to predict the expected effect of consuming a specified media content item or feature (e.g., a song or an acoustic parameter) on a specified health parameter of a population. For example, in cases where consuming a specified media content item (or type of media item) is sufficiently correlated with a change in the value of a specified health parameter in populations that exhibit the media biomarker, it may be predicted that an individual who exhibits the media biomarker and consumes the specified media content item will experience the expected change in the corresponding value of the health parameter.

In some embodiments, media biomarkers may be used to prescribe a media consumption regimen with therapeutic properties. Such therapeutic applications of media biomarkers may be referred to as “health equalizer” applications. For example, media biomarkers may be used to generate a media content playlist (e.g., music playlist) for therapeutic applications. In some cases, the media content playlist may include media content items or features that have a known probability of modulating a parameter related to user's health state (e.g. to maintain a health parameter within a pre-defined range). In some cases, consuming the items on a media content playlist may selectively modulate one or more specified health parameters related to a complex health condition (e.g., modulating heart rate, blood pressure, and/or glucose levels for a diabetic). In some cases, the media content playlist may include media content items for consumption in conjunction with administration of a drug, wherein the consumption of the items on the media content playlist is predicted to extend the drug's effect or reduce the drug's adverse side effect on health state. In some cases, the media content playlist may be determined based, at least in part, on the current health state, currently prescribed drugs, and/or predicted effect of drugs, wherein consumption of the items on the media content playlist is predicted to augment/extend drug action.

In some embodiments, media biomarkers may be used to create or engineer media content items (e.g., music or audio items) that are predicted to have a specified impact on the health state of individuals or populations that exhibit the media biomarker.

In some embodiments, media biomarkers may be used for biofeedback applications. For example, in cases where a media biomarker is sufficiently correlated with a particular health state, a notification may be generated and sent to an individual (or population) when the media biomarker is detected in the individual (or population). As another example, in cases where a media biomarker indicates that consumption of a type of media M is correlated with a health state H, a notification may be generated and sent to an individual who exhibits the media biomarker when the individual consumes the specified type of media.

An example of a biomarker application is identifying music biomarkers indicative of depression in a teen. The music consumption signature underlying the music biomarker may include a greater than 70% frequency of listening to music in the acid rock genre with combined acoustic properties of tempo greater than or equal to 120 bpm, high entropy and high percussion amplification. The association between the music consumption signature and depression may be identified based on correlations between the signature and health data indicative of a depressed health status (e.g. greater than 30% sustained increase in the user's heart rate and electrodermal activity and a score in the 80th percentile or above on the user's response to the clinical Beck Depression Inventory Scale).

Other examples of biomarker applications may include, without limitation, providing media content (e.g., music) based on a personalized physiological parameter (e.g. user's stride length, cadence, etc.) that has a modulating effect on mobility and gait in people suffering from Parkinson's disease, people suffering from movement disorders, and/or stroke patients; using media content (e.g., music) to modulate an individual's social behavior (e.g., to increase propensity for socialization); using sensors to measure stress and anxiety parameters for the purpose of predicting hyper-arousal episodes and meltdowns in conditions like Autism/Dementia and selecting and delivering a personalized playlist to an individual to regulate the level of stress/anxiety; providing media content playlists (e.g., music playlists) with personalized media content signatures (e.g., music signatures) that improve memory, concentration and cognition in adults; using media content (e.g., music) to induce purchasing intent; using media content (e.g., music) to calm a population in a geographical area (e.g., an airport or airplane); using media content to modulate the health state of a population; studying a health condition through media content consumption (e.g., music consumption).

User Interface

The system may generate an interactive user interface (106) having multiple controls and a display area that allows the user to interact with the system (e.g., to input health and contextual data and their associated data acquisition and analysis parameters, and to visualize the music-health-context relationships evaluated by the system). The user interface may allow the user to enable biofeedback to receive personalized media recommendations from the system as well as enable auto-play of suggested playlists via the media player. These user operations may be performed by the user by any suitable device or technique, for example, a touch on a display screen, a verbal command or key command (e.g. a keystroke), a click, or a mechanical switch. Many types of user interfaces known in the art (e.g., touch driven interfaces, voice driven interfaces, in-application, web-application or embedded application type interfaces, and interfaces with static layouts or responsive fluid layouts) are suitable for some embodiments of the present invention. The user interface may display user data and music-health-context relationships in any suitable single or multidimensional, tabular, graphical or dashboard format.

In some embodiments, user interface 106 presents a visualization of an interaction between a population's media consumption (e.g., music consumption) and the population's health. The visualization may be generated by the analysis engine. In some embodiments, generating the visualization includes obtaining health data regarding the health of the population, obtaining media consumption data regarding the media consumption (e.g., music consumption) of the population, synchronizing the health data and the media consumption data to a time series, and mapping the media consumption data and/or the health data to values of visualization parameters. In some embodiments, the visualization shows how one or more of the population's health parameters correlate with the population's media consumption.

In some embodiments, the health data is obtained from the health module. In some embodiments, the health data includes one or more health parameters related to the population's health.

In some embodiments, the media consumption data is obtained from the media module. The media consumption data may include any suitable data that describes attributes or features of media consumed by the population. The media consumption data may be fine-grained (e.g., may relate to a fragment of a media content item, where the fragment may be of any duration) or coarse-grained (e.g., may relate to an entire media content item, collection of media content items, etc.).

The visualization may include a recommendation for one or more members of the population to consume specified media content. Such a recommendation may be included when the visualization is generated in response to determining that the population's health satisfies one or more criteria for a media content intervention. The specified media content may be correlated with a desired change in the population's health. The recommendation may include a recommended duration of the intervention.

For example, a visualization may recommend consumption of media content that is correlated with lower blood pressure in response to detecting that a user's blood pressure is above a threshold level. As another example, a visualization may recommend consumption of media content that is correlated with improved mental health in response to detecting that a user's depression symptoms have worsened by a relative or absolute amount. As another example, a visualization may recommend consumption of media content that is correlated with a desired microbiome profile in response to detecting that a user's microbiome profile differs from the desired microbiome profile by a threshold amount. As another example, a visualization may recommend a decrease in the tempo of music to which a user is listening when the user's heart rate is above a threshold value.

A visualization may include information representing or derived from one or more visualization parameters. Visualization parameters may include, without limitation, color, shape, transparency, location, size, content, and/or any other suitable attribute of text or graphics. In some embodiments, visualization parameters may include coordinates of data points on a graph.

The analysis engine may map data to be visualized (e.g., health parameters and/or media consumption data) to a visualization parameter. The data to be visualized may be directly mapped to the visualization parameter without transforming the data. Alternatively or in addition, data to be visualized may be indirectly mapped to a visualization parameter through application of one or more data transformations. In some embodiments, the data transformations may include any mathematical, computational, or statistical techniques suitable for transforming data (e.g., normalizing data to a relative or absolute scale, converting data to a linear, non-linear or logarithmic scale, mapping data to different types of mathematical progressions (e.g. arithmetic, geometric or Fibonacci progressions) partitioning data into discrete bins, etc.). The data may be mapped to discrete or continuous values of a visualization parameter.

The analysis engine may map health data and media consumption data to different visualization parameters, with or without transformation (e.g., the tempo of music content may be mapped to the color of a graphic, and the heart rate corresponding to the tempo may be mapped to the transparency of the graphic). Alternatively or in addition, the analysis engine may combine health data and media consumption data into a combined parameter/value, and map the combined parameter/value to a visualization parameter (with or without any transformation).

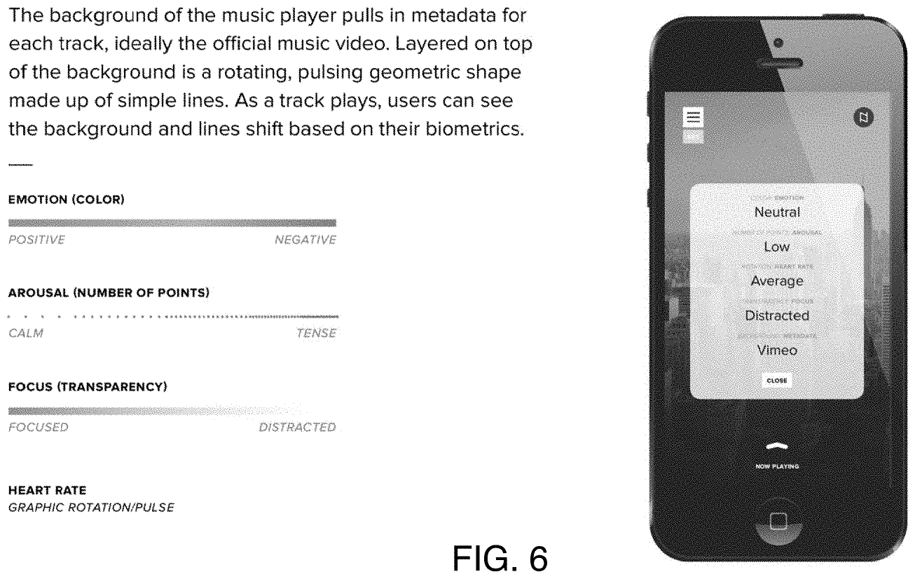

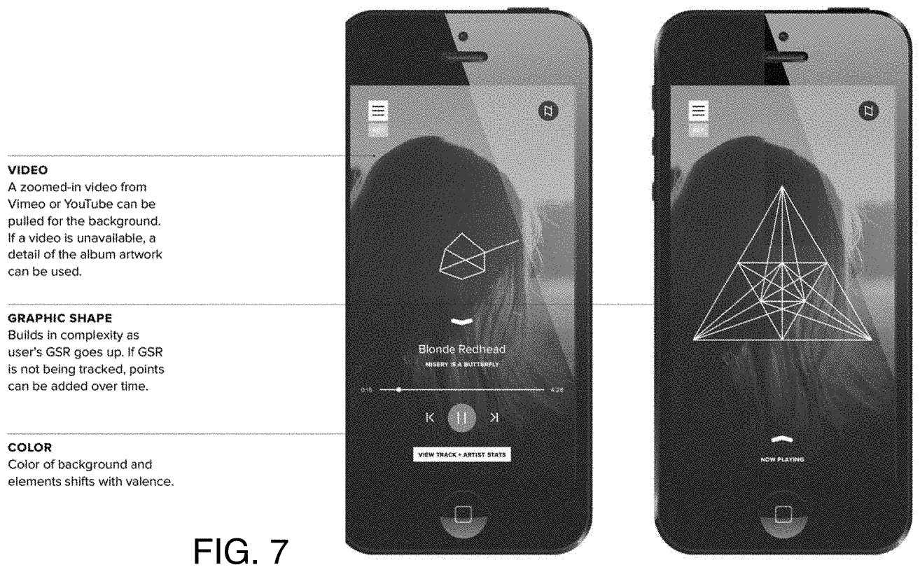

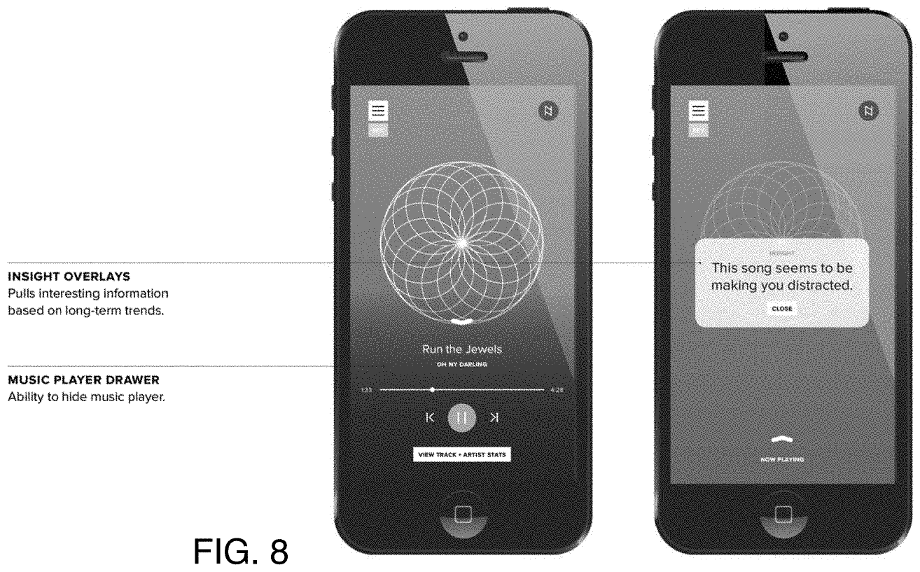

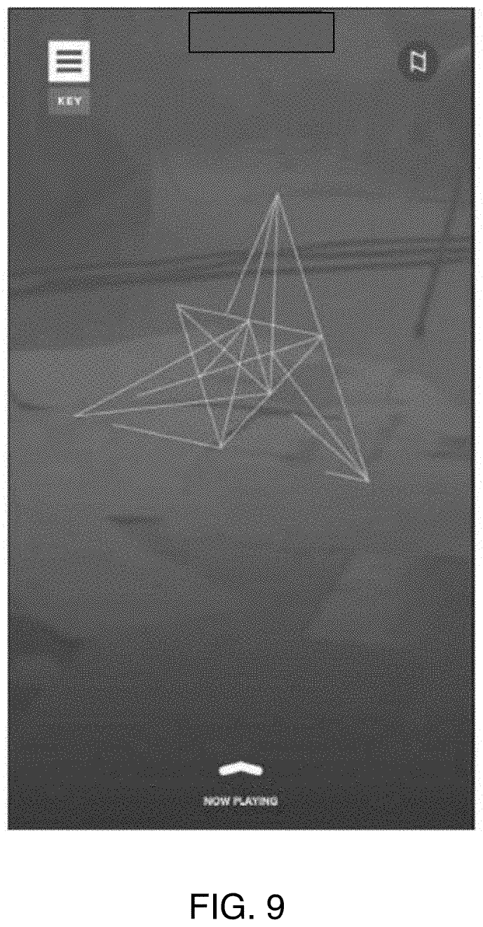

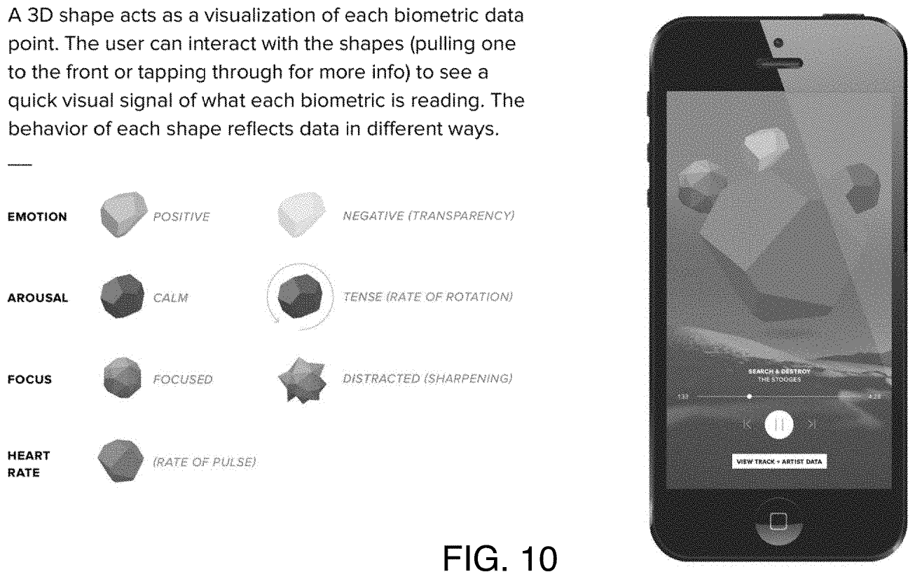

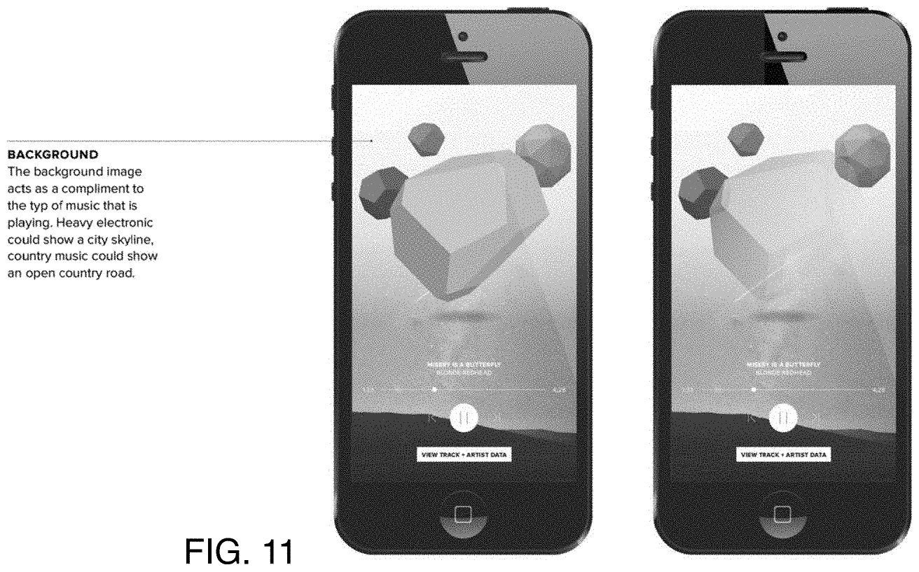

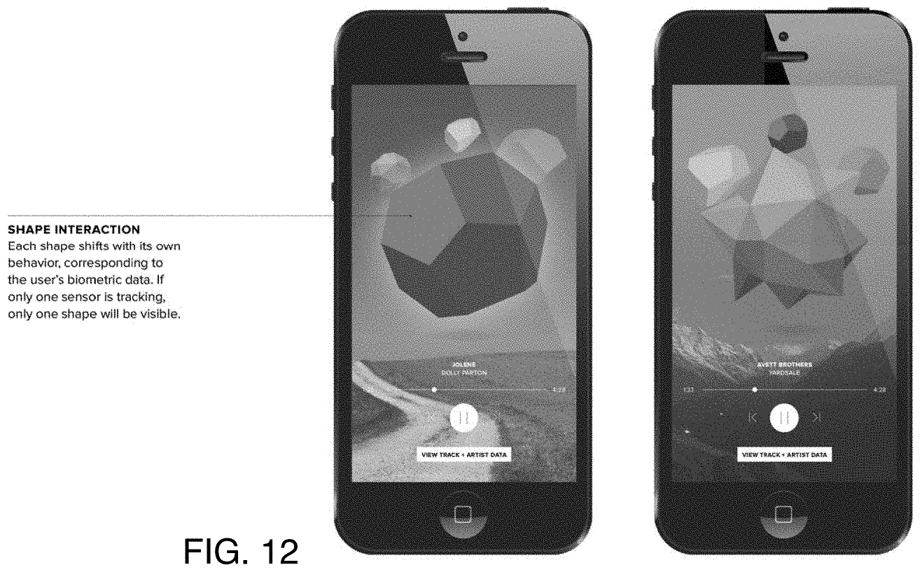

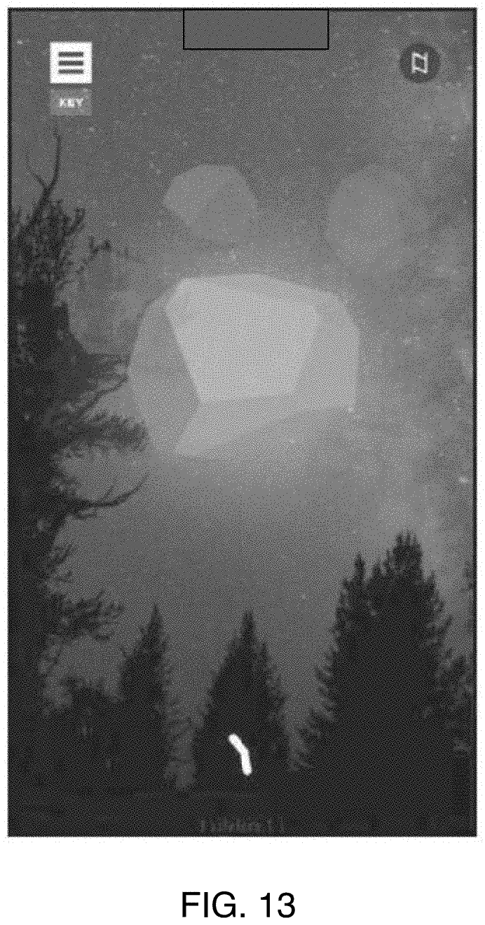

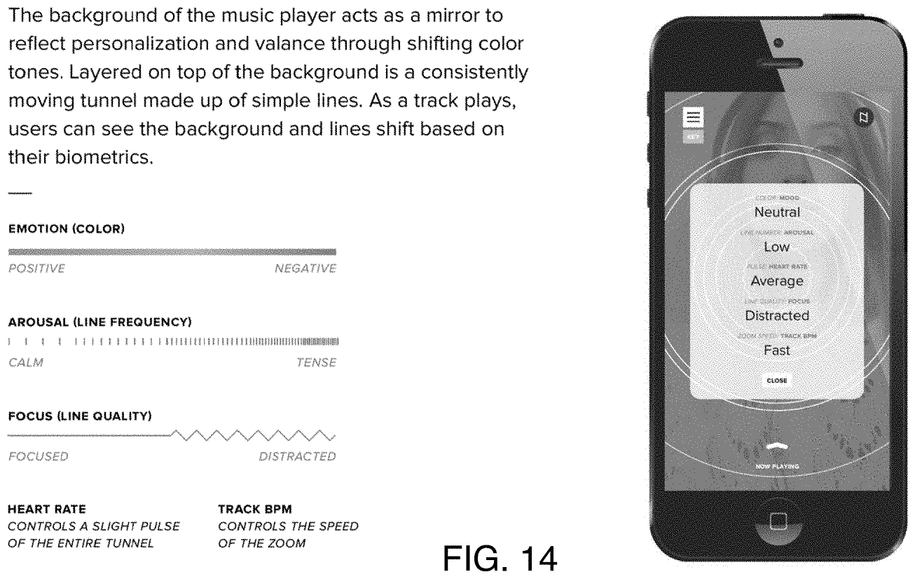

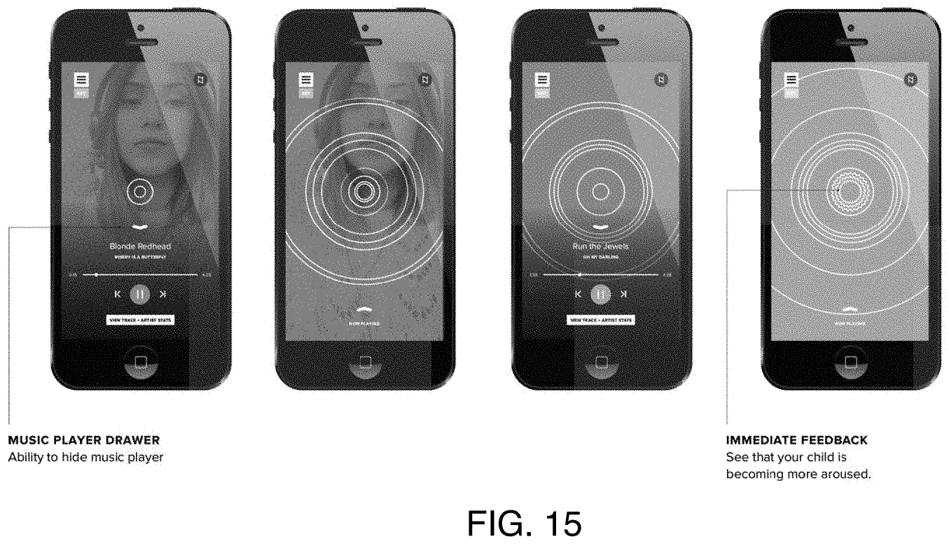

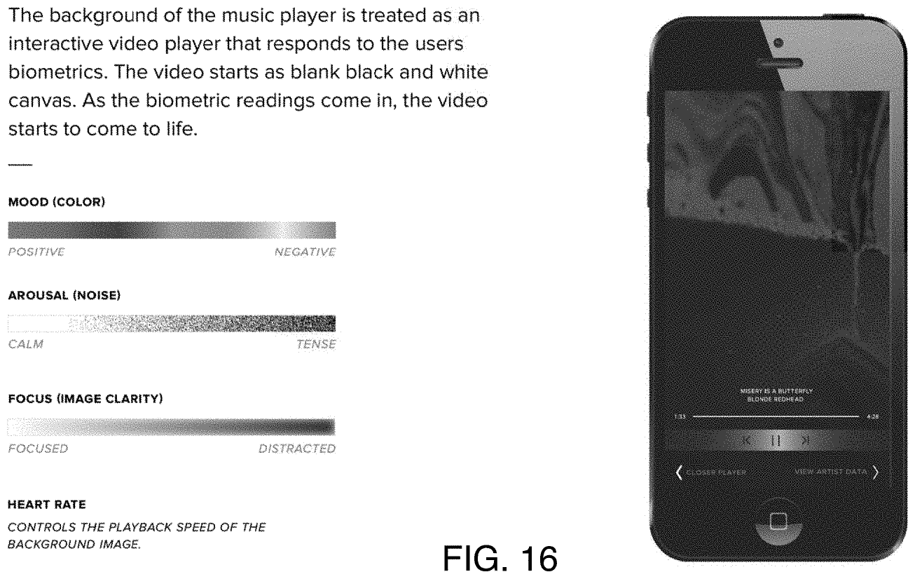

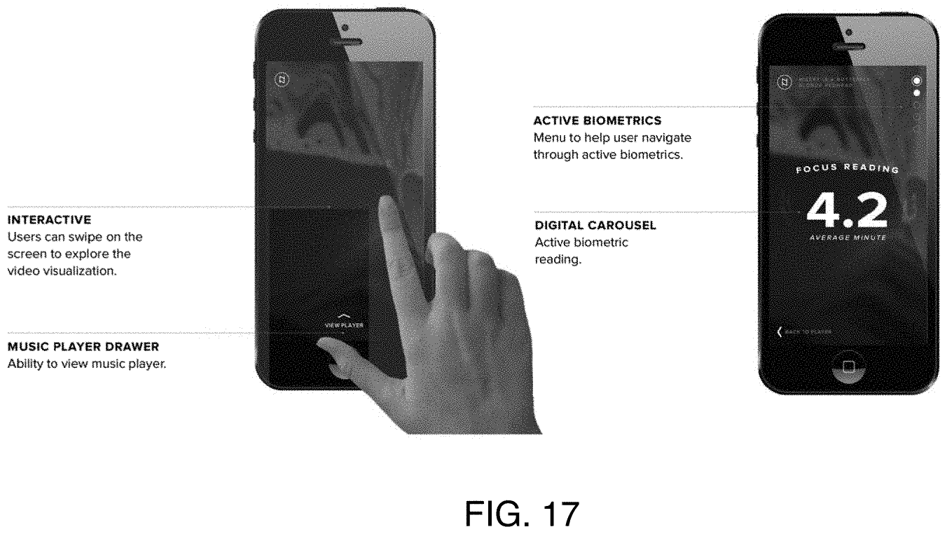

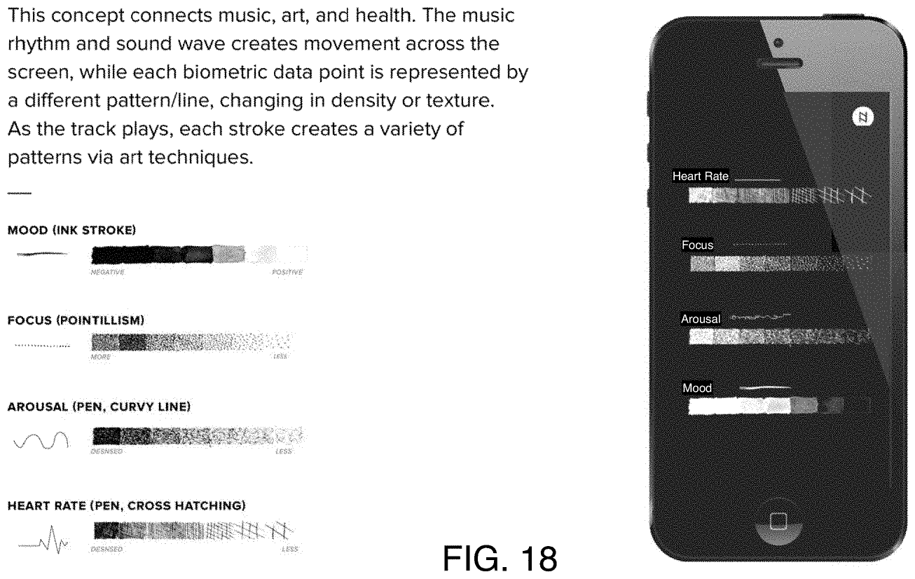

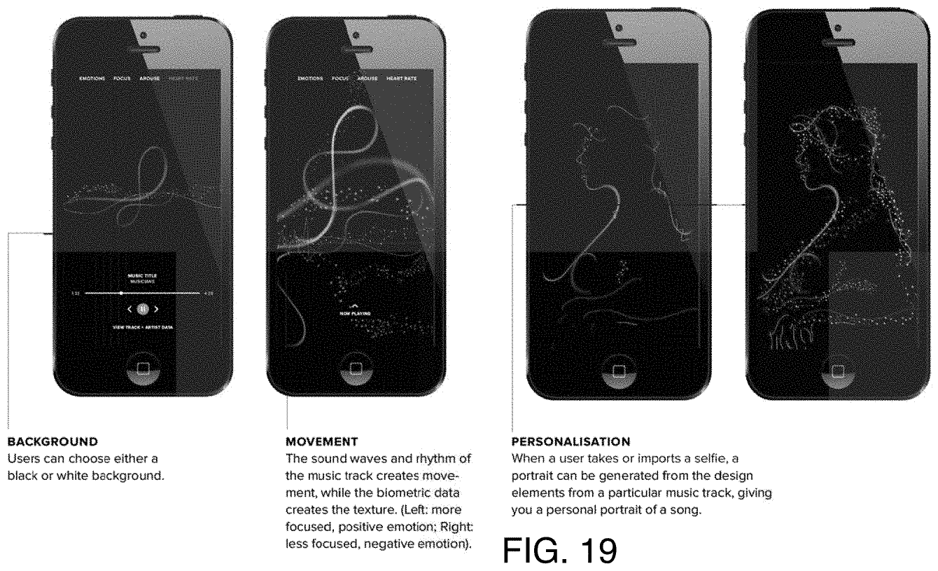

Some exemplary visualizations of health data and/or media consumption data are shown in FIGS. 6-19. In particular, FIGS. 6-9 show exemplary visualizations of health data and/or media consumption data using two-dimensional geometry, in accordance with some embodiments. FIGS. 10-13 show exemplary visualizations of health data and/or media consumption data using three-dimensional geometry, in accordance with some embodiments. FIG. 14-15 show exemplary visualizations of health data and/or media consumption data using tunnels, in accordance with some embodiments. FIGS. 16-17 show exemplary visualizations of health data and/or media consumption data using interaction between liquids, in accordance with some embodiments. FIGS. 18-19 show exemplary visualizations of health data and/or media consumption data using musical paths, in accordance with some embodiments. The visualizations shown in FIGS. 6-19, and the descriptions included in FIGS. 6-19, are given by way of example only. Some embodiments are not limited by the content of FIGS. 6-19.

The user interface may present the visualization using any suitable display technique. In some embodiments, the visualization may be presented on a smartphone screen, a laptop display, a smart watch display, smart glasses, ear buds, earphones, and/or any other suitable display device. In some embodiments, the visualization may be presented on clothing, jewelry, watches, wristbands, shoes, consumer goods (e.g., stuffed animals, key chains, etc.), orbs, totems, etc. In some embodiments, the visualization may be presented on an electronic device via a user interface of software (e.g., a mobile application) executing on the electronic device.

For example, presenting a visualization may include changing the color of ear buds or a light orb as the user's heart rate changes. As another example, mood data may be normalized to a linear scale and mapped to color values (e.g., RGB values) of a displayed graphic, such that the color of the graphic changes as the user's mood changes. As another example, presenting a visualization may include displaying a graphic that uses colors, motion, transparency, shapes, etc. to represent health parameters and/or media consumption data.

Embodiments have been described in which music consumption data and/or health data are mapped to visualization parameters, and visual information regarding interactions between music consumption and health is displayed. In some embodiments, media consumption data and/or health data are mapped to presentation parameters, and sensory information regarding interactions between media consumption and health is presented. Presentation parameters may include, without limitation, visual presentation parameters (e.g., the above-described visualization parameters), auditory presentation parameters, haptic presentation parameters, and/or olfactory presentation parameters. Sensory information may include, without limitation, information that can be sensed by sight (visual information), sound (auditory information), touch (tactile information), smell (olfactory information), and/or taste (taste information). For example, when a user's heart rate is above a threshold heart rate and the tempo of music to which the user is listening is above a threshold tempo, the system may alert the user to the high heart rate/high tempo combination by causing the user's phone or watch to vibrate.

In some embodiments, the system may present sensory information regarding interactions between information sensed by a user and the user's health. In some embodiments, the system may present sensory information regarding the user's health.

In some embodiments, the system may present a visualization of an interaction between the user's context (e.g., environment) and the music (or other media) consumed by the user. The user's context may be represented by one or more contextual parameters. Contextual parameters may include, without limitation, weather parameters (e.g., temperature, humidity, precipitation, cloud cover, etc.), geographical parameters (e.g., location, elevation, inside or outside, etc.), social parameters (e.g., alone or with other people, engaged in conversation or not, in public vs. at home vs. at work vs. in a social setting, identities of nearby people, etc.), time parameters (e.g., date, time, etc.), activity parameters (e.g., what activity is the user engaged in, whether the user is moving, etc.), etc. For example, the system may present a visualization showing the types of music to which the user listens when the user in different environments (e.g., jazz when stuck in traffic, up-tempo music with a certain pitch when it's raining, etc.).

In some embodiments, the system may present a visualization of a user's health data in response to the user's media consumption data meeting specified criteria. In some embodiments, the system may present a visualization of a user's media consumption data in response to the user's health data meeting specified criteria.

In some embodiments, the system may control a device based on an interaction between information sensed by a user and the user's health. For example, when a viewer is consuming media content presented by a smartphone and the user's health data matches specified criteria, the system may control the smartphone to present different media content.

In some embodiments, the system may control a device or material based on the user's health data. The system may control the device by changing a property or behavior of software (e.g. app/phone screen locks, app sends notifications to phone screen or to other devices, etc.), hardware (e.g., robot changes behavior), or a material (e.g., gel becomes harder or softer, gel becomes more opaque or more transparent, gel changes color and/or other properties, etc.). Such materials may, in some embodiments, be incorporated into devices. The system may control the device to provide direct behavioral biofeedback (e.g., regulating behavior or physical state of health devices and/or implantable devices, adjusting settings of pacemaker, sending a signal to an implanted medical device to release anti-stress drugs, etc.).

For example, the above-described presentation techniques may be applied to translucent earphones embedded with light sources (e.g., LEDs). The light sources may be configured to change color when the user is consuming music and the user's heart rate decreases by 5% of a reference value. The user's heart rate readings may be normalized to an initial heart rate value when user starts listening to music. Each 5% interval on the heart rate scale may be associated with a discrete RGB value. As the user's heart rate decreases to a new 5% interval, a signal may be sent to the light sources to change to the RGB value associated with the new interval.

For example, the above-described presentation techniques may be applied to an interactive GUI on a smart device. Media consumption data and health data may be visualized via an interactive graphical user interface (GUI) on the smart device. The device may provide (e.g., play and/or stream) music and record one or more of the user's health parameters (e.g., mood, arousal level, focus, and/or heart rate) while the music is provided. The user's health parameters may be monitored continually, periodically, intermittently, or at any other suitable times. The user's mood data may be transformed linearly and mapped to a continuous color scale (e.g., RGB scale) associated with the background color of the user interface. As the user's mood changes, the background colors may be altered along the color scale. The user's focus data may be processed and binned into discrete numeric intervals, with each interval mapped to a specific transparency value of the user interface. As the focus values switch to a new interval, the transparency of the interface may change to indicate the new focus level. The heart rate data may be processed and binned into intervals, with each interval associated with a specific geometric shape that is displayed on the interface. The arousal level may be normalized to a scale ranging between clinically relevant minimum and maximal values, and the normalized range may then be mapped to an angular scale that specifies the rotational motion of the geometric shape displayed on the user interface. As the arousal level of the user changes in response to the music, the geometric shape displayed on the device screen may rotate through an angle that corresponds to the magnitude of the change in the user's arousal level.

For example, the above-described presentation techniques may be applied to a watch or wristband. The watch or wristband may be configured to vibrate, make a sound, or change temperature as the user's diabetes symptoms and/or glucose levels rise above specified thresholds.

For example, the above-described presentation techniques may be applied to a child's stuffed animal. The stuffed animal may be configured to change colors, make noise, and/or plays music to an infant when the infant's motion increases by a specified amount (e.g., 20%) and respiratory rate increases by a specified amount (e.g., 10 points).

For example, the above-described presentation techniques may be applied to a hearing aid. The hearing aid may be configured to modulate the properties of the music, speech, or ambient sounds being processed by the hearing aid to regulate the user's physiology based on the user's current physiological state. If the hearing aid's user is listening to music and the user's blood pressure is increasing, the hearing aid may alter the sound (e.g., warp the sound or reduces its intensity) to alert the user to the increased blood pressure levels, or to alert the user to switch to music that is correlated with reducing the user's blood pressure levels.

Biofeedback Module

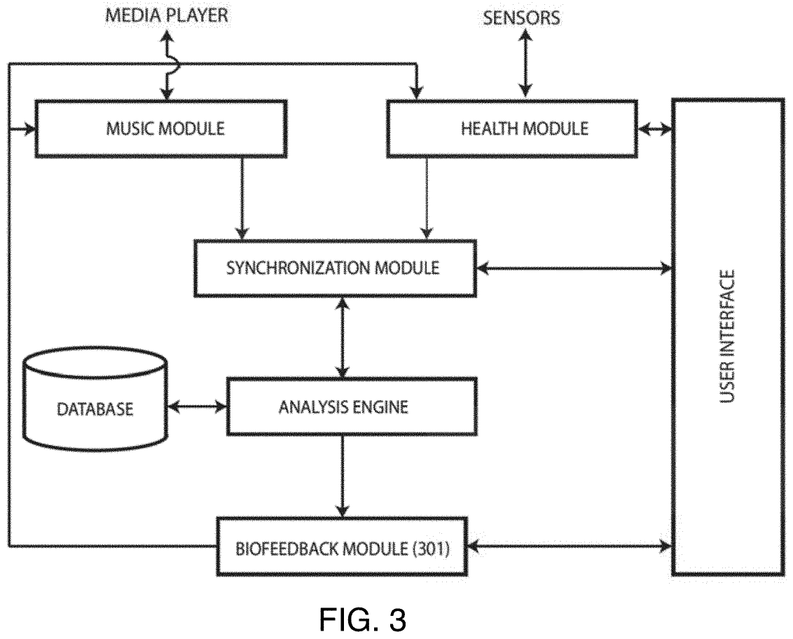

In some specific embodiments, the system may include a biofeedback module that provides personalized media content (e.g., music) recommendations to the user after learning the user's media content preferences in various contexts and the effect(s) of different media content items on user health. In these embodiments, and as shown in FIG. 3, the biofeedback module (301) communicates with the health module to receive data on the user's current health (and, optionally, context). The biofeedback module may refer to the user's personalized media-health-context profile and predictive model generated by the analysis module to assemble and/or suggest media content items for playback to the user using any suitable approach. The system may suggest media content items that match and maintain the user's current health state measured by the health module. Alternately, the system may recommend media content items that are associated with a different user mood or physiology, with the goal of driving the user towards the target health state. For example, when a user is feeling relaxed and wants to maintain this state, the system may recommend media that was effective in maintaining a relaxed state of the user on previous occasions. However, if the user desires to feel energized, the system may recommend media that the user previously consumed in energized states, or before energized states and was measured by the system to be effective in driving the user to this state.

The system may suggest a single media content item or, a media content type comprising a number of media content items, or numerous media content types (e.g. music, videos, movies) and numerous media content items within each type. The system may only suggest media content items stored on a user's local device or may suggest items that can be externally downloaded or streamed via internet based media applications. The system may also obtain the media content items by downloading them from an external source. Suggestions for suitable media content items may be displayed to the user on the user interface. In some embodiments, the biofeedback module interacts with the media module to cause the media player to automatically render the selected media content items.

Recommendations generated by the biofeedback module may be restricted to media content items that have been previously tested by the system for their effect on user health (and, optionally, context). Alternately, the biofeedback module may recommend a new media content item that has not been previously tested, but has metadata and/or features similar to media content items determined to be effective. This allows the user to automatically discover and test new media content that may be pleasurable and effective for regulating their health and activities.

The biofeedback module may generate a signal for the health module to acquire new measurements of user health (and, optionally, context) during or after the presentation of the selected media content to characterize the user's response to the presented item(s). The system may continue to render the selected media content if it is determined to have a positive effect on the user and is effective in driving the user towards the target state. If the media content is ineffective or has a negative effect, the system may use the most recent measurement to select new media content items that are more suitable. Each new measurement may be conveyed to the analysis module to refine the predictive model and to continually improve the media-health-context associations of the user.

It is appreciated that the biofeedback module may not always be engaged by the system. For a new user, the biofeedback module may be enabled after an initial learning phase of a suitable duration, during which time the system first learns the user's media preferences and the associations between the user's media consumption, health and context. In some embodiments, the user may choose to enable the biofeedback module on a pre-set schedule and at specific time-intervals such as once a day or every Monday. In other embodiments, the biofeedback module may be enabled during pre-specified activities or contexts, such as while running or while driving to work. The biofeedback module may also be configured to auto-enable when pre-determined criteria regarding user health and/or context are satisfied. For example, biofeedback may be automatically triggered each time a user's heart rate exceeds 100 beats-per-minute or when the environmental temperature falls below 40 F.

Methods

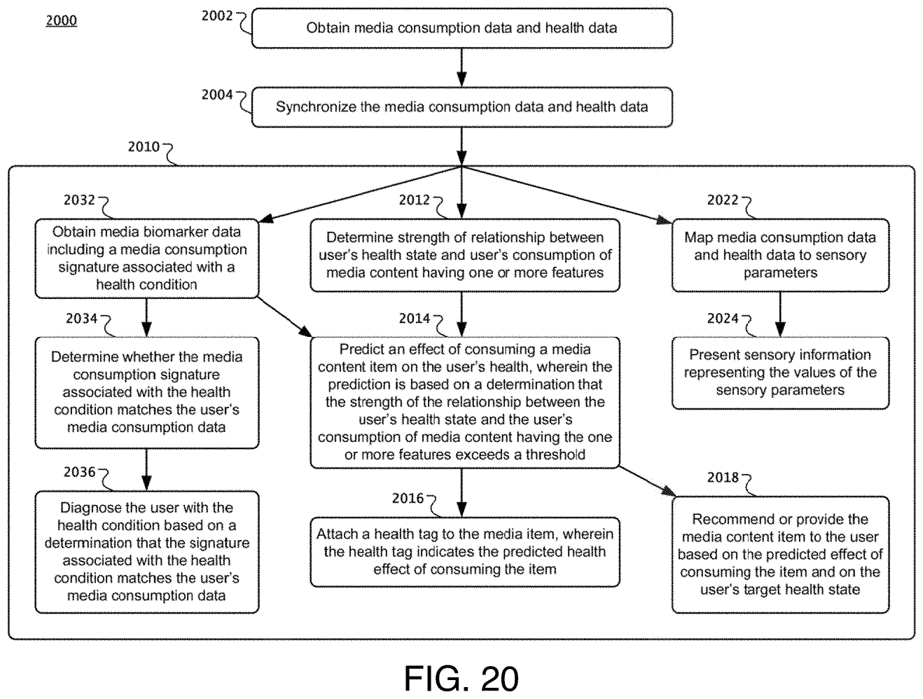

FIG. 20 illustrates a method 2000 for identifying and exploiting relationships between media consumption and health, according to some embodiments. In steps 2002-2004 of method 2000, media consumption data and health data are obtained and synchronized. In subroutine 2010 of method 2000, relationships between media consumption data and health data are identified and/or exploited. The elements of method 2000 are described in further detail below.

In step 2002, media consumption data regarding media content consumed by a user during one or more time periods are obtained. In some embodiments, obtaining the media consumption data includes receiving the media consumption data from one or more sensors (e.g., microphones, cameras, video cameras, etc.). For example, such sensors may be located proximate to the user (e.g., on the user's body, in the user's home, etc.) and/or may be part of an electronic device used by the user (e.g., a smart phone, tablet computer, or laptop computer). In some embodiments, obtaining the media consumption data includes receiving the media consumption data from one or more devices configured to present media content (e.g., televisions, desktop computers, laptop computers, tablets, smart phones, etc.) to the user. For example, such devices may provide access to streaming media services, including but not limited to streaming video services (e.g., Netflix, Amazon Prime, etc.) and streaming audio services (e.g., Audiobooks.com, Pandora, etc.). Other sources of media consumption data are possible, including the sources described above.

The media content consumed by the user may include two or more media content items having one or more same features. In some embodiments, the one or more features include one or more audio features relating to an audio portion of the media content. In some embodiments, one or more of the audio features relate to sound quality of an audio portion of the media content. In some embodiments, one or more of the audio features relate to the harmonic complexity of an audio portion of the media content. In some embodiments, the one or more audio features include one or more low-level audio features of an audio portion of the media content. In some embodiments, at least one of the audio features is a compound audio feature relating to an audio portion of the media content.

In some embodiments, the one or more features include one or more visual features relating to a visual portion of the media content. In some embodiments, visual features relate to a video's representation. Some non-limiting examples of video representation features include the resolution, bit rate, compression, encoding, aspect ratio, frame rate, and/or format of a video. In some embodiments, visual features relate to the content of a video or image. Some non-limiting examples of visual content features include colors, shapes, scenes, objects, people, places, or activities depicted in a video or image. Other types of visual features are possible.

In some embodiments, the one or more features include one or more text features relating to a text portion of the media content. In some embodiments, text features relate to the representation of text. Some non-limiting examples of text representation features include font, font size, and color. In some embodiments, text features relate to the content of text. Some non-limiting examples of text content features include letters, words, or phrases contained in a text, concepts or themes expressed by a text, topics of a text, etc. Other types of text features are possible. Other features of media content items are possible, including the features described above.

In some embodiments, the media consumption data include preference data indicating one or more preferences of the user regarding the media content consumed by the user. A user's preferences regarding the consumed media content may be reported by the user and/or obtained using any other suitable technique. In some embodiments, the user's preferences regarding the consumed media are determined objectively based on the amount or rate of consumption of media content by the user, and/or based on the user's media consumption pattern (e.g., based on changes in the amount of media content or the type of media content being consumed by the user). Other embodiments of media consumption data are possible, including the embodiments described above.

In step 2002, health data are also obtained. In some embodiments, at least a portion of the health data relates to health states of the user during one or more time periods for which media consumption data have been obtained. In some embodiments, obtaining the health data includes receiving the health data from one or more sensors configured to sense the user's health parameters. For example, such sensors may be located proximate to the user (e.g., on the user's body, adjacent to the user's bed or desk, etc.) and/or may be part of an electronic device used by the user (e.g., a smart watch, smart phone, tablet computer, or laptop computer). In some embodiments, obtaining the health data includes loading the health data from a database, receiving the health data from an Internet-based service, or receiving the health data from the user or a healthcare provider (e.g., responses to a survey or clinical scale). Other sources of health data are possible, including the sources described above.

In some embodiments, the health data includes values of one or more health parameters of the user. The health parameters may relate to any aspect of the user's health, including but not limited to the user's physiology, psychology, mood, activity, well-being, and/or behavior. Other types of health parameters as possible, including the health parameters described above.

In some embodiments, method 2000 further includes a step (not shown) in which contextual data is obtained. At least a portion of the contextual data may relate to the user's context during the one or more time periods for which media consumption data and/or health data have been obtained. In some embodiments, the contextual data includes values of one or more contextual parameters. The contextual parameters may relate to any aspect of the user's context, including but not limited to the user's physical environment, the user's temporal environment, and/or the activities in which the user is engaged. Other types of contextual parameters are possible, including the contextual parameters described above. The contextual data may be obtained from one or more sensors (e.g., thermometers, barometers, clocks, motion sensors, etc.), from a database, or from an Internet-based service. Other sources of contextual data are possible.

In step 2004, the media consumption data and the health data are synchronized. In some embodiments, the media consumption data and the health data are time-series data, and synchronizing the data involves aligning the timestamps from the time-series media consumption data and the time-series health data. In some embodiments, the contextual data is also synchronized with the media consumption data and the health data. Some techniques for synchronizing data sets are described above.

In subroutine 2010, relationships between media consumption data and health data are identified and/or exploited. Relationships between media consumption data and health data may be identified and/or exploited in many different ways, corresponding to different paths through subroutine 2010. Some embodiments of various techniques for identifying and/or exploiting relationships between media consumption data and health data are described at a high level here, and discussed in further detail below.

In some embodiments, relationships between media consumption data and health data are identified and/or exploited by presenting sensory information representing a population's synchronized media consumption data and health data. In some embodiments, such sensory information may be presented by performing steps 2022 and 2024 of subroutine 2010. Presentation of such sensory information may, for example, facilitate a user's efforts to identify relationships between media consumption data and health data.

In some embodiments, relationships between media consumption data and health data are identified and/or exploited by predicting the effects of consuming a media content item on a user's health. In some embodiments, such effects may be predicted by performing steps 2012 and 2014 of subroutine 2010, or by performing steps 2012, 2032, and 2014 of subroutine 2010. Such predictions may be used, for example, to provide biofeedback to the user, to prescribe media therapy for the user, and/or to generate health tags for media content items.

In some embodiments, relationships between media consumption data and health data are identified and/or exploited by recommending or providing, to the user, media content items that are predicted to affect the user's health state. In some embodiments, such media content items may be identified by performing steps 2012, 2014, and 2018 of subroutine 2010, or by performing steps 2012, 2032, 2014, and 2018 of subroutine 2010. The user may, for example, consume the identified media content items to maintain a current health state, to achieve a target health state, or to treat a health condition. In some embodiments, the user may consume the identified media content items in connection with a medical intervention or with use of a drug, and consumption of the media content items may enhance the efficacy of the medical intervention or the drug.

In some embodiments, relationships between media consumption data and health data are identified and/or exploited by attaching a health tag to a media content item based on the predicted health effects of consuming the media content item. The health tag may indicate the predicted health effect of consuming the media content item. In some embodiments, such health tags may be attached to media items by performing steps 2012, 2014, and 2016 of subroutine 2010, or by performing steps 2012, 2032, 2014, and 2016 of subroutine 2010. Such health tags may be used, for example, to identify media content items which, when consumed, are predicted to have a particular effect on the user.

In some embodiments, relationships between media consumption data and health data are identified and/or exploited by diagnosing the user with a health condition based, at least in part, on a determination that the user's media consumption data matches a media biomarker for the health condition. In some embodiments, such diagnoses may be obtained by performing steps 2032, 2034, and 2036 of subroutine 2010. Such diagnoses may be used, for example, to screen individuals for further examination and evaluation relating to the health condition.

The individual steps of subroutine 2010 are now described. Although some steps (e.g., steps 2012, 2014, and 2032) are shared by multiple paths through subroutine 2010, repetitive discussion of the steps of subroutine 2010 is avoided in the interest of brevity.

In step 2022, at least a portion of the media consumption data obtained in step 2002 and at least a portion of the health data obtained in step 2002 are mapped to values of one or more sensory parameters. The portions of media consumption data and health data mapped to the sensory parameters may correspond to the same time period. In some embodiments, the sensory parameters correspond to visual, auditory, tactile, olfactory, and/or taste properties of sensory information. Some examples of sensory parameters are described above. Other sensory parameters are possible.

In some embodiments, the media consumption data are mapped to a first visualization parameter, and the health data are mapped to a second visualization parameter. Each of the visualization parameters may control, for example, the color, transparency, shape, rotation, translation, and/or pixilation of a graphic displayed to the user. Other visualization parameters are possible. In some embodiments, the two visualization parameters may be different parameters of the same graphic. In some embodiments, the two visualization parameters may be the same or different parameters of distinct graphics.

In step 2024, sensory information representing the values of the sensory parameters may be presented to the user. The sensory information may include visual information, auditory information, tactile information, olfactory information, and/or taste information. Some techniques for presenting sensory information are described above. Other techniques for presenting sensory information are possible.

In some embodiments, correlation data regarding a correlation between a portion of the media consumption data and a portion of the health data are generated and mapped to a sensory parameter. Mapping correlations between media consumption data and health to sensory parameters may facilitate identification of strong correlations by the user.

In step 2032, media biomarker data are obtained. The media biomarker data includes one or more media biomarkers. Each media biomarker may include data identifying a media consumption signature and a health state (or health condition) associated with the media consumption signature. In some embodiments, a media biomarker may include data indicating a strength of an association (e.g., correlation) between the media consumption signature and the health state. In some embodiments, a media biomarker may include data indicating whether the biomarker is diagnostic or predictive. Predictive biomarkers indicate that media consumption consistent with the biomarker's media consumption signature is predicted to drive the user's health state toward the health state identified by the biomarker. Diagnostic biomarkers indicate that the presence of the biomarker's media consumption signature in the user's media consumption data is predictive of the user having the health condition identified by the biomarker.

Obtaining the media biomarker data may include loading the data from a memory device or receiving the data over a computer network. Alternatively or in addition, media biomarker data may be generated. Generating media biomarker data may involve obtaining health data regarding the health of a population and obtaining media consumption data regarding media consumption of the population. The population's media consumption data may include a media consumption signature. Generating the media biomarker data may further involve generating relationship data regarding a relationship between the media consumption signature and a portion of the population's health data corresponding to a health condition. If the strength of the relationship exceeds a threshold strength, a media biomarker may be generated. The generated biomarker may include data identifying the media consumption signature, the associated health condition, and the strength of the association between the signature and the health condition.

A media consumption signature may include one or more media consumption characteristics of a population. In some embodiments, media consumption characteristics include the amount of media consumed by the population (e.g., frequency and duration of media consumption), the rate at which media is consumed by the population, a range of media consumption amounts or rates, one or more media consumption preferences of the population, and/or one or more patterns of media consumption by the population. Some embodiments of media consumption signatures and characteristics are described above. Other media consumption signatures and characteristics are possible. In some embodiments, the media consumption characteristics of a signature may apply to media consumption generally, or to consumption of particular types of media content (e.g., media content within a particular content category, or media content having particular features).

The relationship or association between a media consumption signature and a health condition may be a correlation. Techniques for determining the strength of correlation (e.g., tests of the statistical significance of a correlation) between a media consumption signature and a health condition are described above. Other techniques for determining the strength of correlation are possible.

In step 2034, a determination is made as to whether the media consumption signature associated with the health condition (the biomarker's signature) matches the media consumption data for the user. Determining whether the biomarker's signature matches the media consumption data for the user may involve identifying one or more media consumption signatures in the user's media consumption data and comparing the biomarker's signature with the user's signatures. In cases where the signatures include media consumption patterns, the patterns may be compared using any suitable pattern matching technique. In cases where the signatures include media consumption amounts, rates, or ranges, the signatures may be compared using any suitable numerical comparison technique. Exact identity between the two signatures may not be required to determine that the signatures match. In some embodiments, it may be determined that two signatures match when the signatures are sufficiently similar.

In step 2036, the user is diagnosed with the health condition indicated by the media biomarker based, at least in part, on a determination that the biomarker's signature matches the user's signature. The diagnosis may also be based, for example, on the results of medical diagnostic tests. In some embodiments, subroutine 2010 also includes a step (not shown) of prescribing a therapy for the user based, at least in part, on the determination that the biomarker's signature matches the user's signature. Prescribing the therapy may involve recommending that the user consume one or more specified media content items. In some embodiments, the therapy may be prescribed in connection with a drug prescription or a medical intervention.

As described above, portions of the media consumption data obtained in step 2002 may correspond to two or more media content items having one or more same features. In step 2012, the strength of the relationship between the user's health state and the user's consumption of media content having those features is determined, based at least in part on the portions of media consumption data corresponding to the user's consumption of the media content items having those features. The determination may also be based, at least in part, on the synchronized health data, the contextual data, and/or the user's media preferences. Some techniques for determining the strength of a relationship (e.g., a correlation) between a health state and media content consumption are described above. Other techniques are possible.

In step 2014, a prediction is made as to how consumption of a media content item is likely to affect a user's health. The prediction may be based, at least in part, on a determination (made in step 2012) that there is a sufficiently strong association between the user's health state and the user's consumption of media content having the one or more features. In some embodiments, the association is determined to be sufficiently strong if the strength of the association exceeds a threshold strength. In some embodiments, the prediction is based, at least in part, on a determination that the signature of a biomarker (e.g., a predictive biomarker) matches a signature in the user's media consumption data. In some embodiments, the prediction is based, at least in part, on one or more user preferences relating to the media content and/or to the one or more features of the media content.

In some embodiments, the prediction is based, at least in part, on a determination that there is a sufficiently strong association between a population's health state and the population's consumption of media content having the one or more features. In some embodiments, the association is determined to be sufficiently strong if the strength of the association exceeds a threshold strength. In some embodiments, the population consists entirely of users other than the user to whom the prediction pertains. In some embodiments, the population includes the user to whom the prediction pertains and other users. In some embodiments, the population includes a single user, such that the prediction is personalized to the user.

In some embodiments, the media content data obtained in step 2002 does not include the media content item to which the prediction pertains. Thus, using the techniques described herein, the impact of consuming a media content item on a user's health may be predicted even if the user has never consumed the media content item before or if media consumption data corresponding to the user's consumption of the item has not been used to make the prediction. In some embodiments, the media content item to which the prediction pertains has the one or more features that were shared by two or more media content items corresponding to the media consumption data. In some embodiments, the predicted effect on the user's health includes a predicted change in the user's intent to purchase certain goods or services.

The predicted effects of consuming the media content item may include long-term effects on the user's health and/or short term effects on the user's health. In some embodiments, short-term effects include effects that are immediately detectable upon the user consuming the media item, or detectable within seconds, minutes, or hours (e.g., up to approximately eight hours) after the user consumes the media item. In some embodiments, long-term effects include effects that are detectable at least a specified time period (e.g., approximately eight hours) after the user consumes the media item. In some embodiments, an effect on the user's health is “detectable” if the corresponding health parameters can be detected using specified health sensors or medical tests.

In step 2016, a health tag is attached to the media content item to which the prediction of step 2014 pertains. The health tag may be attached to the media content item as metadata of the media content item. In some embodiments, the health tag includes information indicating the predicted health effect of consuming the media content item. The predicted health effect may be specific to one or more users (personalized), or may apply to a population. In some embodiments, the predicted health effect includes a predicted change in a user's (or population's) intent to purchase certain goods or services.

In step 2018, one or more media content items are recommended or provided to the user for consumption. The media content item(s) may be selected based, at least in part, on a prediction that consuming the item(s) will maintain the user's current health state or facilitate a transition to a target health state. In some embodiments, the selection of the media content item(s) is based, at least in part, on health tags associated with the one or more media content items. For example, the health tags may indicate that the media content items are suitable for maintaining the user's current health state or driving the user to a target health state. In some embodiments, the selection of the media content item(s) is based, at least in part, on the strength of the relationship between one or more features of the item(s) and the user's current or target health state. In some embodiments, the selection of the media content item(s) is based, at least in part, on the strength of the relationship between a pattern of media consumption by the user and the user's current or target health state. For example, the selected media content item(s) may be arranged such that consuming the media content item(s) (e.g., in a recommended sequence) yields the pattern of media consumption.

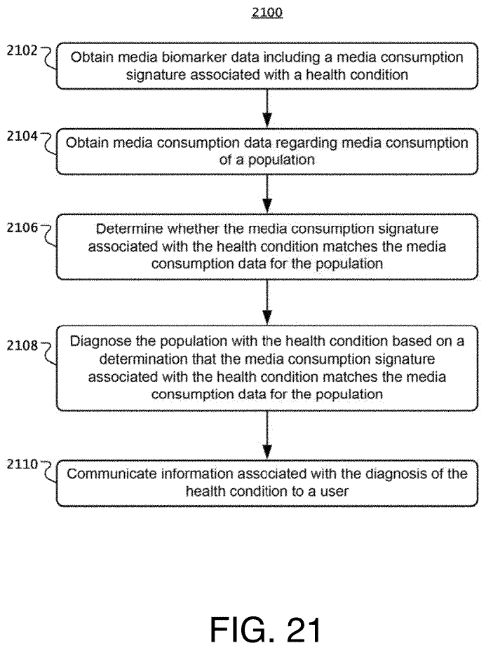

FIG. 21 illustrates another method 2100 for identifying and exploiting relationships between media consumption and health, according to some embodiments. The method 2100 may be used to diagnose the existence of a health condition in a population.

In step 2102, media biomarker data are obtained. The media biomarker data include one or more media biomarkers (e.g., diagnostic media biomarkers). Each media biomarker may include data identifying a media consumption signature and a health state (or health condition) associated with the media consumption signature. In some embodiments, a media biomarker may include data indicating a strength of an association (e.g., correlation) between the media consumption signature and the health state. Techniques for generating or otherwise obtaining media biomarkers are described above. In some embodiments, the media biomarker data are provided by a research tool. Some embodiments of media consumption signatures are described above.

In step 2104, media consumption data are obtained. The media consumption data may correspond to media consumption of a population. In some embodiments, the population corresponding to the media consumption data is the same population whose media consumption data and health data were used to generate the media biomarker data. In some embodiments, those two populations differ. Either population may consist of a single person, or may comprise two or more people (e.g., people who have one or more characteristics in common). In cases where the population consists of a single user, the media consumption data and the analysis thereof is personalized to the user.

In step 2106, a determination is made as to whether the signature of a media biomarker matches the media consumption data of the population. Techniques for determining whether a media consumption signature matches a set of media consumption data are described above.

In step 2108, a diagnosis is made. The population may be diagnosed with the health condition associated with the media biomarker based, at least in part, on a determination that the biomarker's signature matches the population's media consumption data. In some embodiments, the diagnosis is also based on other data, including but not limited to the population's health data and/or results of medical tests administered to members of the population,

In step 2110, information associated with the diagnosis of the health condition is communicated to a user. Communicating the information may include displaying the information, causing the information to be displayed, presenting the information audibly (e.g., using text-to-speech synthesis), causing the information to be presented audibly, and/or transmitting the information (e.g., over a communication network).

In some embodiments, the method 2100 further includes a step of predicting, based at least in part on the determination that the biomarker's signature matches the population's media consumption data, a health effect (for a member of the population) of consuming a particular item of media content. In some embodiments, the method 2100 further includes a step of attaching a health tag to a media content item as metadata of the media content item based, at least in part, on a determination that the media content item matches the biomarker's signature. The health tag may include information indicating that consumption of the media content item is associated with the health condition corresponding to the biomarker.

In some embodiments, the method 2100 further includes a step of prescribing a therapy for a member of the population, based at least in part on the determination that biomarker's signature matches the population's media consumption data. In some embodiments, the prescribed therapy includes consuming particular items of media content (e.g., a recommended set or sequence of media content items). In some embodiments, the prescribed therapy also includes administration of a drug or performance of a medical intervention in connection with the consumption of the particular items of media content. In some embodiments, consuming the media content items may improve the efficacy of the drug or medical intervention.

In some embodiments, the status of the health condition in the population is monitored by repeatedly (e.g., periodically, intermittently, at scheduled times, at randomly selected times, etc.) obtaining new media consumption data for the population and determining whether the biomarker's signature matches the population's new media consumption data.

Some Target Groups and Applications

Some embodiments can be implemented as personalized monitoring platforms to continuously track and analyze users' media consumption patterns through a broad set of activities and environments. Individuals that can use these platforms can be any person, especially those interested in monitoring their general media consumption and gaining insights into their media preferences in different contexts. All types of media consumers ranging from music and other media aficionados to occasional recreational media users can benefit from the platform to evaluate their consumption of different types of media, music by certain artists, specific albums or songs during their daily activities, as well as to identify their favorite artists, genres and media pieces. Such platforms would be particularly beneficial to individuals interested in evidence-based selection of media, and in improved personalized playlist generation to match specific activities and environments. For example, students can use the platform to identify media items that improve their focus and concentration, or memory processing for more efficient learning. Similarly, elderly users can identify media content that improves their sleep quality, and athletes can identify media that improves their performance in an exercise or sport. Such individuals and groups can further benefit from embodiments of the present invention that employ a biofeedback mechanism to automatically recommend and render suitable media content to effect the desired health state during a current activity.

The present disclosure describes methods to measure the effect of individual media items on a person's health. As such, some embodiments can identify and render media content that is efficacious in achieving a specific health state of a user. Some embodiments can therefore be implemented as a personalized therapeutic platform that can be used by individuals as a self-therapy tool to manage non-clinical conditions in daily living, for example to regulate their mood, stress, pain and overall activity and wellbeing. Platforms that have been customized for, and validated in clinical populations can be prescribed by therapists and medical professionals as primary or adjuvant therapeutic interventions in various clinical indications. There are numerous potential populations that can benefit from prescribed therapeutic media platforms, including but not limited to patients that have been diagnosed with clinical depression or anxiety, cardiovascular problems, sleep disorders, fatigue, chronic and acute pain, mental disorders (e.g., Alzheimer's disease), movement disorders (e.g., Parkinson's disease), patients in post-surgical and post-stroke recovery, and patients with other clinical conditions in which media content may have a therapeutic effect.

Other individuals that can benefit from some embodiments include biomedical and life science researchers, clinicians and therapists who can employ the disclosed system as a research platform to test the effect of specific media content on human health and physiology and/or to identify media biomarkers indicative of health states. For instance, the platform can be employed to test how a genre of music or a specific song affects cardiovascular parameters or activates the emotional, motor or other centers in the brain. Similarly, the techniques disclosed herein have utility as a commercial and marketing research platform, and would be of value to the media content industries for assessing the effects of new media content on users. As an illustrative example, music composers can employ the commercial research platform to test how listeners respond to a new music composition, or to evaluate which of multiple alternate music compositions is most effective in inducing a desired emotional response or physiological state in listeners. As another example, movie producers could deploy multiple versions of a movie trailer to sub-groups of platform users to determine which trailer elicits a positive user response and increases (e.g., maximizes) the movie or ticket purchasing behavior of the target population. Similarly clinics, hospitals, airport waiting areas, restaurants, shops, departmental stores and other commercial institutions that use music and other media to create a specific ambience can utilize some embodiments of the present invention to determine which media content is most effective in generating a desired mood and mental state in their clients and customers. Some embodiments can further be employed to identify and stratify categories, clusters or subgroups of users having similar health responses to a media content type or item for participation in a clinical or commercial research study. User stratification can additionally aid targeted marketing applications, where some embodiments can deploy marketing media content customized to specific subgroups of users who have been determined to have a positive response to media items of similar types and content. For example, the platform may advertise an upbeat song from a music album to 20 year old individuals that have been determined to respond positively to energetic music, and showcase another song with lower beats per minute and less rhythmic variation from the same album to users in their 60s in order to increase (e.g., maximize) purchase of the album by both groups.

Some embodiments be incorporated into multimedia and virtual environments to create immersive user experiences. For example, the platform can be deployed in an interactive computer game to personalize and adapt the gaming environment to the player's current physiological and mental state. As another example, virtual classrooms may employ the platform to continuously test the mental state of attendees and customize the presented media content to improve or maintain class attention and learning.

### Example 1: Personalized Platform to Track Music Preferences and Consumption Pattern

We have designed and built a prototype personalized media, health and environment tracking platform according to aspects of the present disclosure. The platform operates as a software application on smartphone devices and mobile tablets. It interfaces with music streaming software applications on the user's device to gather data on the attributes and features of songs streamed to the user. The platform interfaces with the accelerometer and gyroscope of the mobile device to obtain data on the user's activity level, steps, pace etc. Activity data is also accrued from software applications that interface with wearable activity monitors such as a Fitbit® band. Data on a user's heart rate, respiration rate, blood pressure, skin temperature and electrodermal skin activity is gathered from interfacing suitable heart rate, respiration and BP monitors, and sensors that measure skin conductance; weather and seasonal data is collected from weather software applications installed on the device, and location data is obtained from the inbuilt device GPS. A user can enter additional information of the type not collected from the interfacing applications and devices (e.g. his/her mood and type of current activity such as reading, running etc.) by directly entering the information through the user interface.

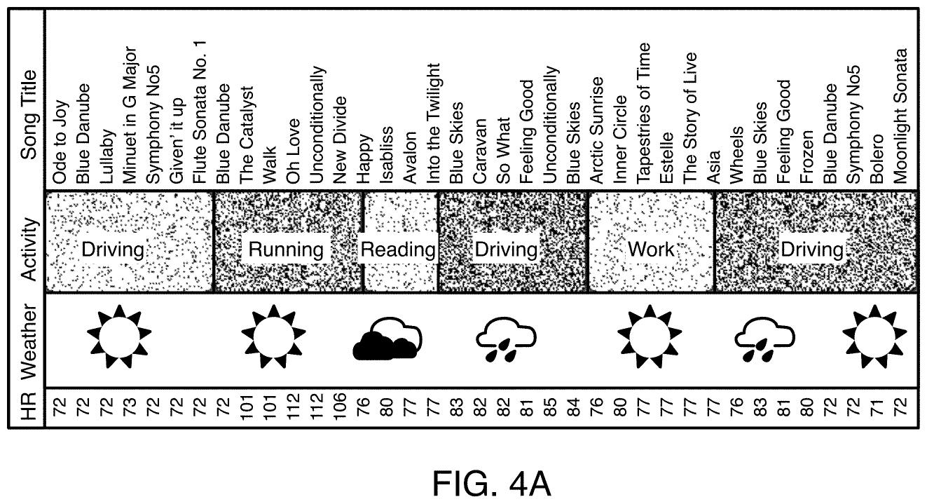

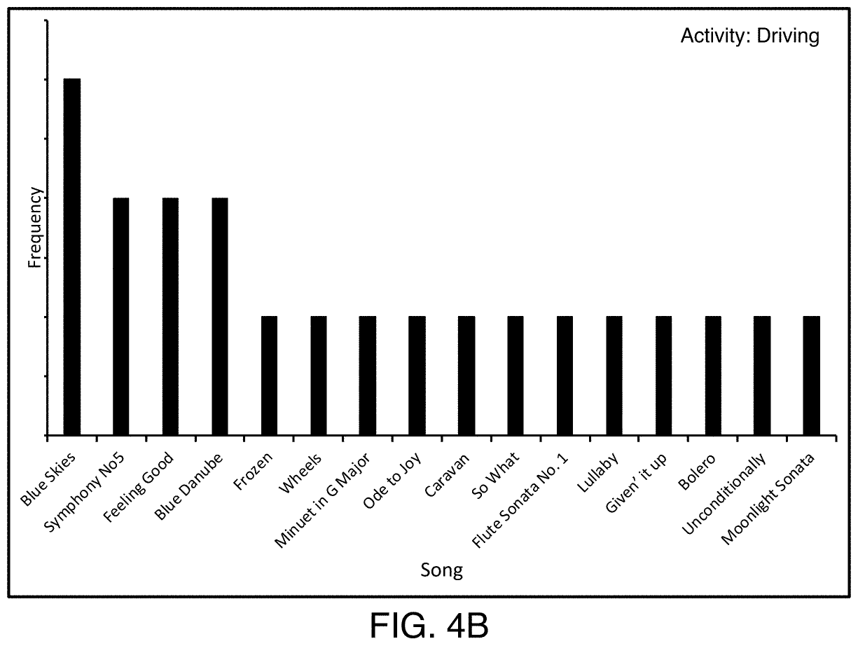

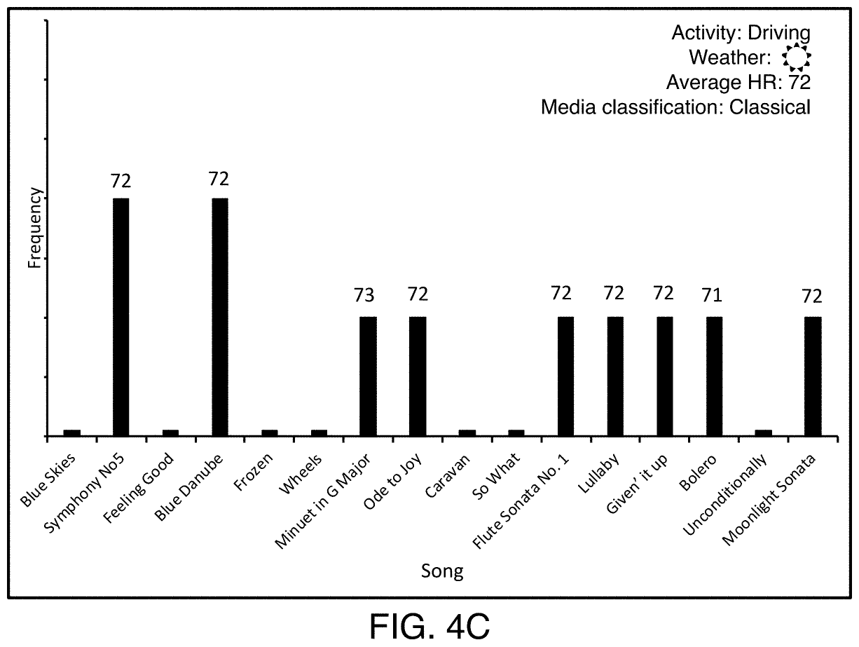

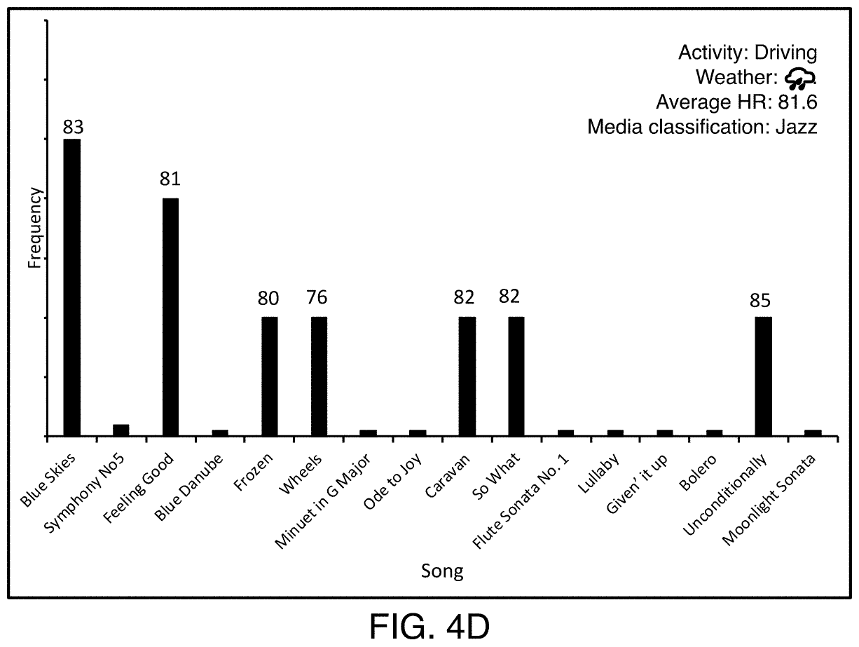

FIG. 4 illustrates data from an exemplary user of the prototype platform. The platform tracks the user's music consumption pattern, weather and heart rate (HR) throughout his daily activities, and the user enters information regarding the type of activity he is engaged in through the application user interface (FIG. 4A). The platform analyses the user's frequency of streaming various types of music and individual songs, and identifies correlations between music and user activity, heart rate and weather. FIG. 4B shows the user's tracked data that has been filtered on the activity parameter to selectively display music that was streamed when the user was driving. By conducting a frequency analysis and constructing histograms of song utilization, the platform determined that the user listens to Strauss's “Blue Danube”, Beethoven's “Symphony No 5”, Nina Simone's “Feeling Good” and Ella Fitzgerald's “Blue Skies” more frequently while driving than rock or pop music. The platform further implemented regression analysis on the media and weather data, and found that the user listens to the classical pieces “Blue Danube” and “Symphony No 5” while driving on a sunny day; on rainy days he plays the jazz pieces by Nina Simone and Ella Fitzgerald (FIG. 4C). Regression analysis between the media and physiology data revealed that listening to classical music correlates with an average heart rate (HR) of 72 bpm for this user, while listening to jazz music correlates with a higher average HR of 81.6 bpm. Therefore, the platform continuously tracked the user's music consumption pattern and determined the user's media preferences to be classical music while driving on a sunny day and jazz music when driving on a rainy day, and the user's HR to be higher when he listened to jazz music.

### Example 2: Clinical Music App with Biofeedback for Cardiovascular Regulation

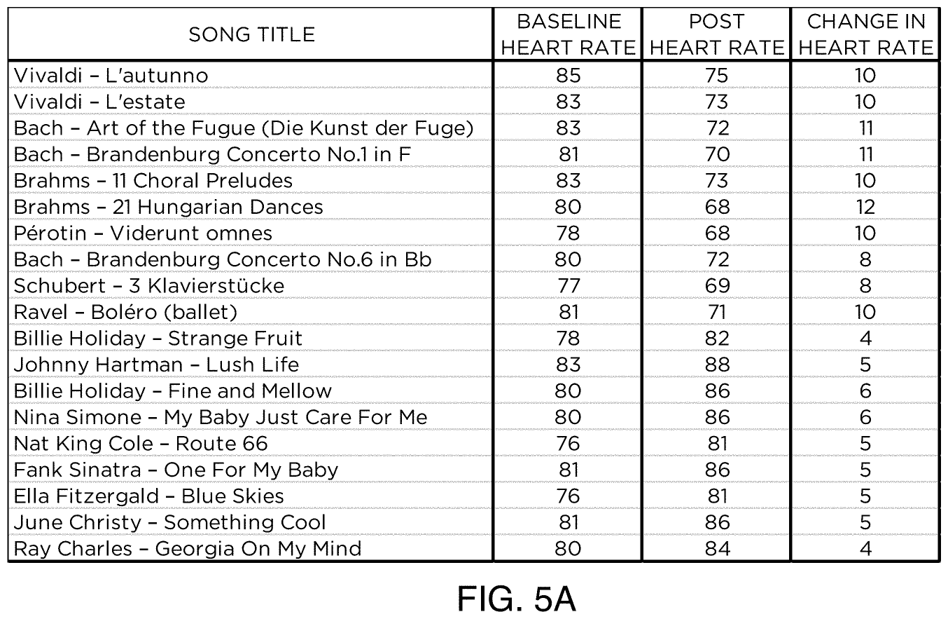

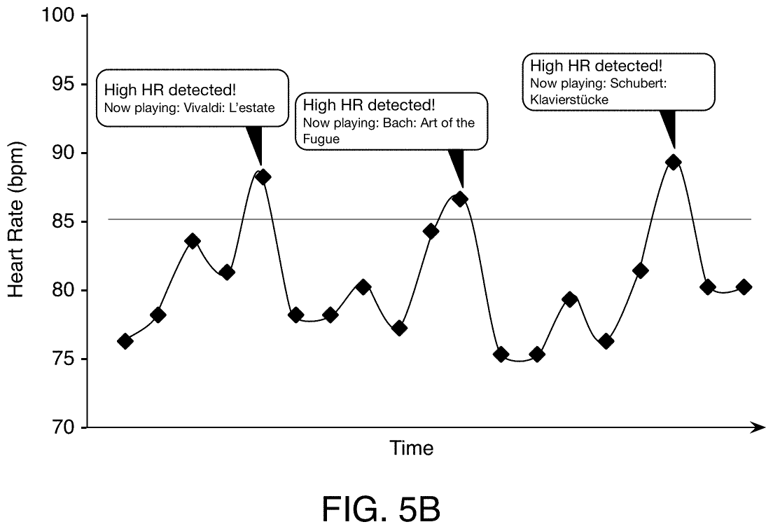

The prototype platform can be customized as a therapeutic clinical tool for regulating cardiovascular parameters. The platform is deployed as a prescribed software app on a patient's smartphone or mobile tablet. When a new user activates the app, it first initiates a learning phase of suitable duration. During this phase the app first tracks the patient's music consumption data and pattern through his/her various activities. It evaluates the effects of the consumed media on the patient's cardiovascular parameters by measuring his/her heart rate and blood pressure before, during, and after he/she listens to each song. These measurements are incorporated into a personalized media-health profile and predictive health model of the patient, and are used to provide suitable media feedback to the patient in the second phase. The second phase is a biofeedback phase, during which the platform continuously monitors the patient's cardiovascular parameters and recommends/renders songs measured to improve these parameters when they fall outside a predetermined normal range for the patient. FIG. 5A tabulates data from an exemplary patient. The platform determined that listening to classical music produced an immediate decrease in the patient's heart rate by an average magnitude of 10 beats per minute. On the other hand, listening to jazz music increased his heart rate by an average of 5 beats per minute. During the biofeedback phase, the app continuously monitored the patient's heart rate and blood pressure and automatically rendered classical music to the patient when his heart rate rose above the predetermined threshold of 85 bpm (FIG. 5B).

### Example 3: Discovery Platform for Personalized Health and Music Biomarkers for Anxiety

The prototype platform can be deployed as a discovery application on a user's smartphone or mobile tablet. The application runs continuously and passively in the background on the user's device and tracks the user's music consumption pattern and health data (e.g., heart rate, blood pressure, respiration rate, electrodermal activity and skin temperature) throughout his/her daily living. The user additionally inputs information on periods of high anxiety through the user interface. The user's health and music consumption data collected via the health and media modules of the application are time synchronized and conveyed to the analysis engine for evaluation of patterns and associations in the data. The analysis engine first processes individual data streams to filter out noise and anomalies by applying entropy and energy based filters. When the quality of data is determined to be greater than a pre-specified threshold value, the analysis engine implements machine learning and statistical analysis techniques comprising feature extraction, covariate analyses and ANCOVA linear regressions on the health and music consumption data to identify patterns that are significantly associated with the ‘anxious’ user health state. In an exemplary user, the analysis engine identified that during episodes of high anxiety, the user's heart rate and blood pressure increased by 10% and respiration rate increased by 20% over baseline values recorded prior to the period of anxiety. Additionally, there was greater than one degree increase in the user's skin temperature and his electrodermal skin activity signal increased sharply by over three times the average daily maximum value and was sustained at these high levels for a period of at least ten minutes. It therefore classified these combined synchronous changes in the health data to be indicative of the anxious health state. The analysis engine further determined that there was a significant correlation between episodes of high anxiety and a 50% increased preference for rock music with characteristics of tempo equal to 160-180 beats per minute, high syncopation and polyphonic texture for this user. Therefore, it characterized ‘a 50% increased preference for rock music with tempo of 160-180 beats per minute, high syncopation and polyphonic texture’ as the music biomarker associated with high anxiety for the user. The personalized music biomarker for anxiety was recorded in the user's music-health profile. In subsequent use of the platform, the analysis engine was able to intelligently determine that the user was in an anxious health state exclusively from music consumption data, when his music consumption pattern matched the personalized music biomarker identified for this condition.

### Example 4: Embedded Application for Immersive Gaming Experience

The prototype platform can be deployed as an embedded software application in a commercial 3D videogame to provide gamers a more immersive gaming experience. A player participates in the game in the form of a virtual avatar that has to complete certain tasks to advance to the next level. The player wears sensors and stands on a mat that collectively monitor his motor movements, which are then translated into movements performed by his avatar. For example, he jogs on the mat to make his avatar run, or moves his hand to direct his avatar to perform a similar hand movement. The player additionally wears an activity and heart rate monitor during the game session. These monitors interface with the embedded media and health tracking system of the present disclosure, which continually tracks the player's steps, pace and heart rate, and also interfaces with the game's media module. The game initiates with a training session, during which the player is presented different media content from the game's media database and his health responses to the presented media content are measured, analyzed and recorded. This is followed by actual gameplay, during which the embedded application rapidly selects and customizes the media presented to the player depending on his/her avatar's current activity and environment in the game. For example, if the avatar is running, the embedded application selects and presents media content that has been determined to increase the pace and heart rate of the user, so he experiences a physiological state that is commonly elicited while running. Alternately, the embedded application presents media content that is measured to reduce the player's heart rate and induce a relaxed state when his avatar is sleeping or relaxing. Therefore, the embedded application interfaces between the physiology and activity of the real user and the activity and environment of his virtual game avatar to create an engaging and immersive gaming experience.

An Implementation

Implementations of the subject matter (e.g., methods) and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The methods and operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both.

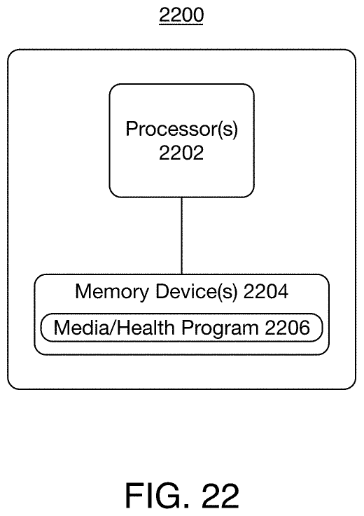

FIG. 22 shows a block diagram of a computer 2200. The elements of the computer 2200 include one or more processors 2202 for performing actions in accordance with instructions and one or more memory devices 2204 for storing instructions and data. Generally, a computer 2200 will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In some embodiments, a computer 2200 or a computer system executes a media/health program 2206. The media/health program may implement the methods and operations described in the present disclosure. Different versions of the media/health program may be stored, distributed, or installed. Some versions of the software may only some embodiments of methods for identifying and exploiting relationships between health data and media consumption data. For example, some versions may implement only certain steps of subroutine 2010 or certain paths through subroutine 2010. Some versions of the software may allow an operator to control which embodiments of the techniques described herein are performed on a data set. For example, an operator may select one or more settings corresponding to particular embodiments of the techniques described herein, and the software may then execute the steps of subroutine 2010 that correspond to the specified embodiments. Multiple embodiments of the techniques described herein may be performed in sequence or in parallel. For example, the software may execute steps of subroutine 2010 forming multiple paths through subroutine 2010 serially or in parallel.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

A number of implementations have been described. Nevertheless, it will be understood that additional modifications may be made without departing from the scope of the inventive concepts described herein, and, accordingly, other implementations are within the scope of the following claims.

What is claimed is:

1. A system for inducing a physical state in a user, comprising:
a sensor associated with the user;
an audio output device; and
a computer configured to:
obtain, from a database, an identification of a type of music associated with a first phase of sleep;
determine, based on data from the sensor, that a user is in a second phase of sleep;
select music of the type associated with the first phase of sleep; and
output the selected music to the user through the audio output device.

2. The method of claim 1, further comprising:
obtaining, from a sensor associated with the user, updated health condition data, the health condition data indicating the user's progress towards the first phase of sleep;
based on the updated health condition data, modifying the selected music; and
outputting the modified music to the user.

3. The method of claim 2, wherein obtaining the updated health condition data comprises monitoring the user's respiration rate.

4. The method of claim 1 wherein the identification of a type of music associated with a first phase of sleep comprises a genre of music.

5. The method of claim 2 wherein the identification of a type of music associated with the first phase of sleep comprises data describing acoustic attributes of music.

6. The method of claim 5 wherein the acoustic attributes of music comprise one or more of beat, tempo, and pitch.

7. The method of claim 6 wherein modifying the selected music comprises reducing the tempo of the music.

8. The method of claim 1 wherein determining that the user is attempting to fall asleep comprises monitoring a user's biometric data and time of day.
